(19) United States
(12) Patent Application Publication
(10) Pub. No.: US 2018/0182489 A1
(43) Pub. Date: Jun. 28, 2018

(54) MEASURE-BASED CHAINING OF NOTIFICATIONS

(71) Applicant: KONINKLIJKE PHILIPS N.V., EINDHOVEN (NL)

(72) Inventors: AKI SAKARI HÄRMÄ, EINDHOVEN (NL); SILVIA BERTAGNA DE MARCHI, EINDHOVEN (NL); KOEN THEO JOHAN DE GROOT, SEVENUM (NL); RIM HELAOUI, EINDHOVEN (NL)

(21) Appl. No.: 15/844,992

(22) Filed: Dec. 18, 2017

Related U.S. Application Data
(60) Provisional application No. 62/438,513, filed on Dec. 23, 2016.

(51) Int. Cl. G16H 50/30; G06F 9/54; G16H 20/30; H04L 12/707; G06F 17/30

(52) U.S. Cl. CPC G16H 50/30; G06F 9/546; G06F 17/3061; H04L 45/22; G16H 20/30

(57) ABSTRACT

In an embodiment, a system is disclosed that receives a plurality of data structures that comprise contextually-related notifications, associates the data structures to starting, intermediate, and ending nodes, establishes plural possible pathways from the starting and intermediate nodes, each of the starting and intermediate nodes comprising a statement table with plural statements (and hence paths) to choose from based on measures, and based on continual input data and computation of the measures of each statement table, adding nodes to link to a selected starting node and then other nodes to ultimately provide a chain of notifications presented in non-overlapping time intervals and in narrative form that provide an indication of progress in advancing a user towards a goal.

### CROSS-REFERENCE TO RELATED APPLICATIONS

This patent application claims the priority benefit under 35 U.S.C. § 119(e) of U.S. Provisional Application No. 62/438,513 filed on Dec. 23, 2016, the contents of which are herein incorporated by reference.

### FIELD OF THE INVENTION

The present invention is generally related to health and wellness monitoring and logically-linked notifications.

### BACKGROUND OF THE INVENTION

Data structures, including programming instructions and/or notifications (e.g., statements), may be executed by one or more processors to provide helpful feedback in control processes and/or monitored user activity. For instance, in the area of health monitoring, such data structures may be used to provide alerts and/or suggestions for purposes of health and wellness programs. In WO2014059390A2, a platform for providing wellness assessments and recommendations using sensor data is described. In one disclosed example, a wearable device is described that may be configured to detect a user's movement between areas and access a database using the location data to provide one or more messages associated with wellness assessment and wellness recommendations. The messages may be ordered in priority or importance. The assessment may be comparisons of user activity for different time periods, and the messages may include recommendations to boost activity levels (e.g., by suggesting engagement of activity on additional days).

### SUMMARY OF THE INVENTION

In one embodiment, a system is disclosed that receives a plurality of data structures that comprise contextually-related notifications, associates the data structures to starting, intermediate, and target nodes, establishes plural possible pathways from the starting and intermediate nodes, each of the starting and intermediate nodes comprising a statement table with plural statements (and hence paths) to choose from based on measures, and based on continual input data and computation of the measures of each statement table, adding nodes to link to a selected starting node and then other nodes to ultimately provide a chain of notifications presented in non-overlapping time intervals and in narrative form that provide an indication of progress in advancing a user towards a goal.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which a notification system is used in accordance with an embodiment of the invention.

FIG. 2 is a schematic diagram that illustrates an example wearable device in which all or a portion of the functionality of a notification system may be implemented, in accordance with an embodiment of the invention.

FIG. 3 is a schematic diagram that illustrates an example electronics device in which all or a portion of the functionality of a notification system may be implemented, in accordance with an embodiment of the invention.

FIG. 4 is a schematic diagram that illustrates an example computing system in which at least a portion of the functionality of a notification system may be implemented, in accordance with an embodiment of the invention.

FIG. 5 is a schematic diagram that illustrates example building blocks for generating a predetermined quantity of statements for a notification system, in accordance with an embodiment of the invention.

FIG. 6 is a schematic diagram that illustrates an example statement family with one or more templates for a notification system, in accordance with an embodiment of the invention.

FIG. 7 is a schematic diagram that illustrates an example statement family with one or more statements for a notification system, in accordance with an embodiment of the invention.

FIGS. 8A and 8B are schematic diagrams of example paths of starting, intermediate, and target nodes with statements that are established by a notification system, in accordance with an embodiment of the invention.

FIG. 9 is a schematic diagram that illustrates an example of candidate chains of notifications used by a notification system, in accordance with an embodiment of the invention.

FIG. 10 is a schematic diagram that illustrates an example processing model for path tracking for a notification system, in accordance with an embodiment of the invention.

FIG. 11 is a schematic diagram that illustrates an example statement table used by a notification system, in accordance with an embodiment of the invention.

FIGS. 12A-12C are schematic diagrams that illustrate an example method for a notification system in accordance with an embodiment of the invention.

FIGS. 13A-13E are screen diagrams that illustrate example notifications presented to a user by a notification system in accordance with an embodiment of the invention.

FIG. 14 is a flow diagram that illustrates an example notification method, in accordance with an embodiment of the invention.

FIG. 15 is a flow diagram that illustrates another example notification method in accordance with an embodiment of the invention.

### DETAILED DESCRIPTION OF EMBODIMENTS

Disclosed herein are certain embodiments of a notification system, apparatus, and method (herein, also collectively referred to as a notification system) that comprises a collection of nodes or data structures that are arranged according to a plurality of possible different paths or chains. After the selection of a starting node (e.g., based on computed measures), as input data is received, measures (e.g., scores) for the nodes indicated in a statement table of the starting node are computed, and measures that meet a predefined criteria (e.g., greater than other scores and/or greater or equal to a predetermined threshold, for instance as determined by historical data or an administrator or programmer) are used to select the next node (and hence next statement table and next statement). Note that selection criteria for the next node may include the above and one or more additional criteria, including personalized criteria, features that meet historical criteria, or based on context aware features. In some embodiments, the scoring may be omitted and the selection of the next node based on one or more of the above-mentioned additional criteria. Stated otherwise, as a statement is selected from among plural statements listed for a given node statement table, the statement is provided (e.g., published to a user), input data is received over an interval of time, and measures are computed for the node statement table to select a next node statement for publication as a notification, and so on until a target node is reached. Thus, each node is added over time to progressively build a chain of nodes. In the meantime, each notification that is presented to the user is a logical step towards influencing a user in reaching his or her goal. When the collection of notifications, though published in temporally different intervals, are viewed as a whole, the resulting chain of notifications comprise a narrative. That is, the statements or notifications comprise a logical relationship and provide a user-perceivable trend or direction towards a goal. The on-going narrative provides continual, logical feedback and/or encouragement in assisting and/or advising the user in advancing progress towards a given goal.

Digressing briefly, health programs typically contain education and feedback content which is usually selected from a limited collection of pre-scripted texts. The quality and limited personalization of the input data (e.g., content feed) is generally understood as one important reason for historically low customer engagement and low interest in the long-term use of such health and wellness services. A large part of the content the user is exposed to is of limited relevance for the user. Certain embodiments of a notification system address the problem of limited user engagement/interest by providing a progressively expanding chain of notifications (e.g., statements, including insights) to the user that collectively form a logical story, a narrative, which may help the user to reach the desired targets.

Having summarized certain features of a notification system of the present disclosure, reference will now be made in detail to the description of a notification system as illustrated in the drawings. While a notification system will be described in connection with these drawings, there is no intent to limit notification systems to the embodiment or embodiments disclosed herein. For instance, though described in the context of health management services, certain embodiments of a notification system may be used to improve engagement of a user in other contexts, including financial management, business management, and industrial control processing. Also, though emphasis is on the publication of a statement after each scoring computation, in some embodiments, scoring may be based on chains of statements to determine which of the possible chains to present. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

Note that throughout the specification, statements and notifications are used interchangeably, the use of the term notification herein generally signifying that the statement is presented electronically to a user. In one embodiment, notifications may comprise a statement, wherein the statement comprises a reference to user data and a behavioral goal of the user and optionally a user preference. Statements are in the form of data structures, and according to the present disclosure are configured to convey information related to a plausible observation directed to the behavior of the user. The statements according to the present disclosure may be further configured to convey health-related information of the user. The statements may be presented as a fact that the user already recognizes. Further, the statements may be presented as a revealing of a hidden behavior pattern with advice to the user to change behavior to a better direction (e.g., improve health). The present disclosure describes a system and method for automatically generating a large number of statements that are meaningful in a particular program context and selecting nodes to link to a preceding node from among plural possible predefined pathways, wherein when viewed from the collective outcome of the various published statements over a period of time, the resulting chain that is based on the selected nodes provides insight to the user (as presented at a user device) based on the node arrangement in narrative form. Measures or scores (those two terms likewise used herein interchangeably, the scores generally a subset of measures) are used both in the initial defining of the statements, the selection of a starting node, and in the selection of subsequent nodes that are linked to form a narrative to be presented to the user. In one embodiment, the measures or scores are computed for each statement of a statement table for a selected node, the scores based on statistical and heuristic weighting rules, and statements are selected that have high scores (and meet a threshold value) for use in plural paths.

During a data structure definition stage, the statements are generated based on user input, which includes dynamic data collected from a particular program implemented on a wearable device as well as long-term observations of a large population of users. The program according to the present disclosure is an application designed to be implemented on a mobile device (e.g., user device) to monitor physiological or psychological signs of the user as well as to track the real-time activities of the user. The program may be a health-related program or a health-related application. The programs developed for those devices employ one or more recommender-type systems to analyze the profile of the user, provide various types of messages to the user, or recommend one or more resources to the user. The statements individually comprise one or more personalized insights of the health-related behavior of the user. The statements may be presented as one or more texts displayed or played on the user device, one or more graphical illustrations displayed on the user device, a content card comprising one or more texts displayed on the user device, a content card comprising integrated texts and graphical illustrations displayed on the user device, or any combinations thereof. The statements may be generated with respect to different objectives, for example, education, feedback on performance, insight, motivation, etc. A statement provides valuable feedback and inspiration to the user, and helps the user to choose new opportunities to form healthier behavior and habits. Accordingly, the notification system can provide to the user, insightful information that is personalized for each individual user and has more impact on the behavior of the user.

Note that use of the terms, node or nodes, refers to logical constructs for describing the data structures and facilitating an understanding of the chaining of the data structures that comprise the statements.

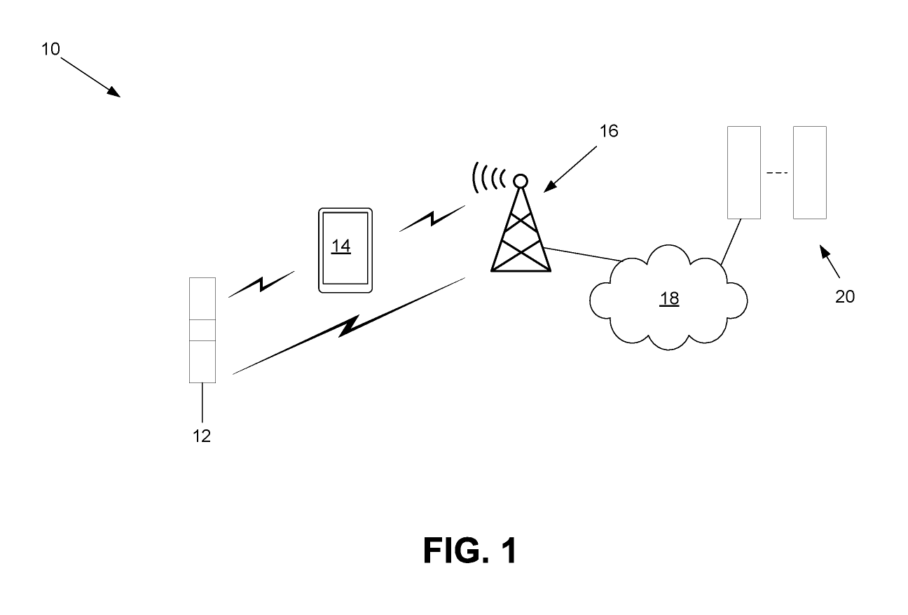

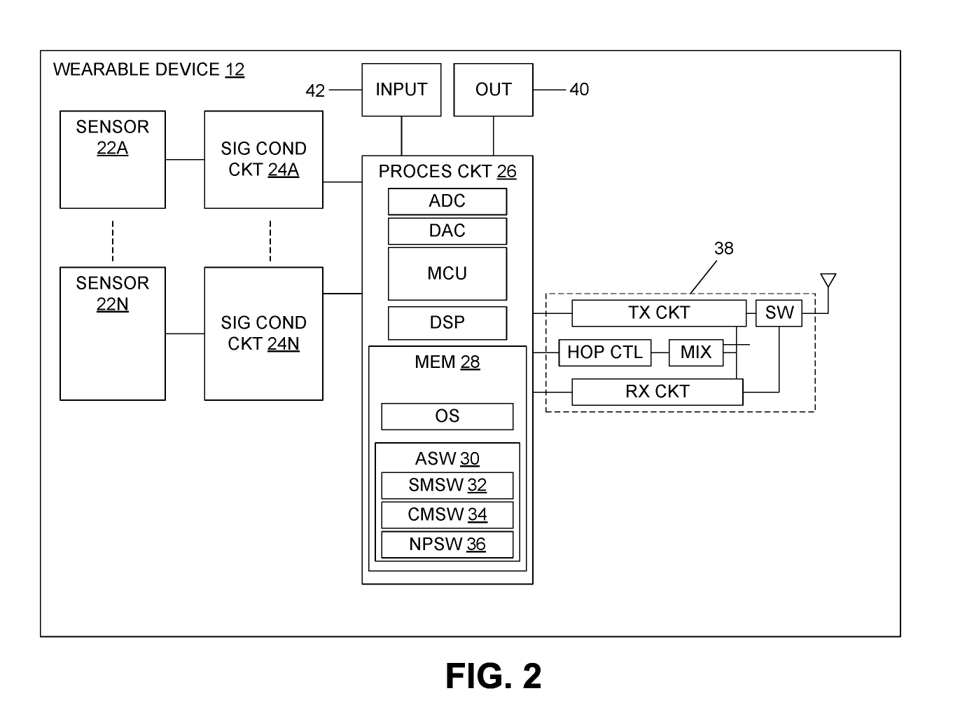

Referring now to FIG. 1, shown is an example environment 10 in which certain embodiments of a notification system may be implemented. It should be appreciated by one having ordinary skill in the art in the context of the present disclosure that the environment 10 is one example among many, and that some embodiments of a notification system may be used in environments with fewer, greater, and/or different components that those depicted in FIG. 1. The environment 10 comprises a plurality of devices that enable communication of information throughout one or more networks. The depicted environment 10 comprises a wearable device 12, an electronics (portable) device 14, a cellular network 16, a wide area network 18 (e.g., also described herein as the Internet), and a remote computing system 20. Note that the wearable device 12 and the electronics device 14 are also referred to as user devices. The wearable device 12, as described further in association with FIG. 2, is typically worn by the user (e.g., around the wrist or torso or attached to an article of clothing), and comprises a plurality of sensors that track physical activity of the user (e.g., steps, swim strokes, pedaling strokes, etc.), sense/measure or derive physiological parameters (e.g., heart rate, respiration, skin temperature, etc.) based on the sensor data, and optionally sense various other parameters (e.g., outdoor temperature, humidity, location, etc.) pertaining to the surrounding environment of the wearable device 12. For instance, in some embodiments, the wearable device 12 may comprise a global navigation satellite system (GNSS) receiver, including a GPS receiver, which tracks and provides location coordinates for the device 12. In some embodiments, the wearable device 12 may comprise indoor location technology, including beacons, RFID or other coded light technologies, WiFi, etc. Some embodiments of the wearable device 12 may include a motion or inertial tracking sensor, including an accelerometer and/or a gyroscope, providing movement data of the user. A representation of such gathered data may be communicated to the user via an integrated display on the wearable device 12 and/or on another device or devices.

Also, such data gathered by the wearable device 12 may be communicated (e.g., continually, periodically, and/or aperiodically, including upon request) to one or more electronics devices, such as the electronics device 14 or via the cellular network 16 to the computing system 20. Such communication may be achieved wirelessly (e.g., using near field communications (NFC) functionality, Blue-tooth functionality, 802.11-based technology, etc.) and/or according to a wired medium (e.g., universal serial bus (USB), etc.). Further discussion of the wearable device 12 is described below in association with FIG. 2.

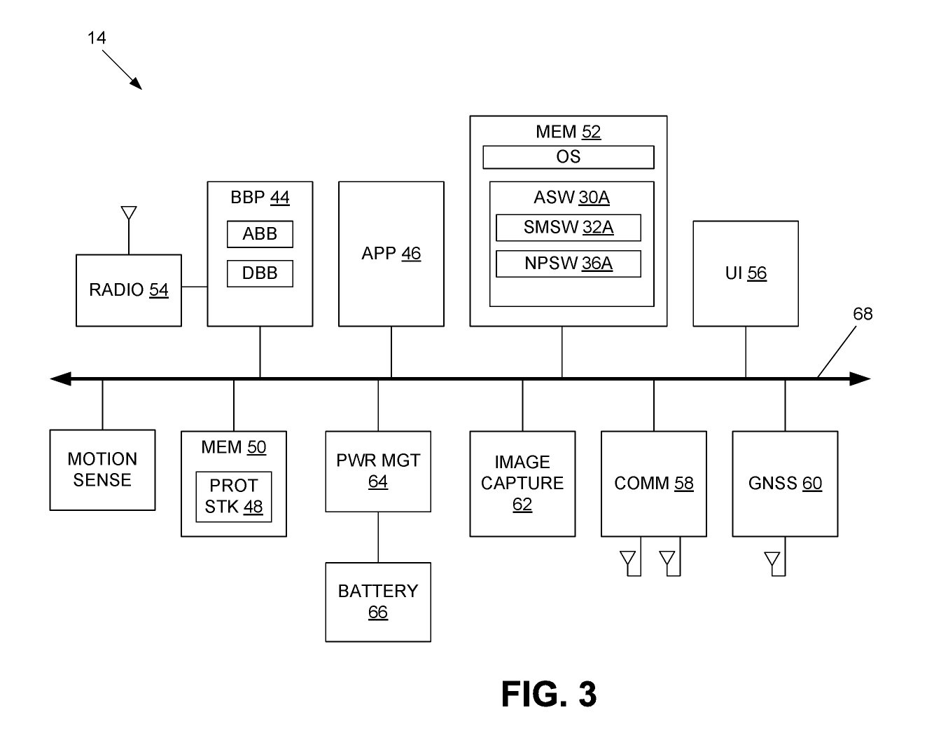

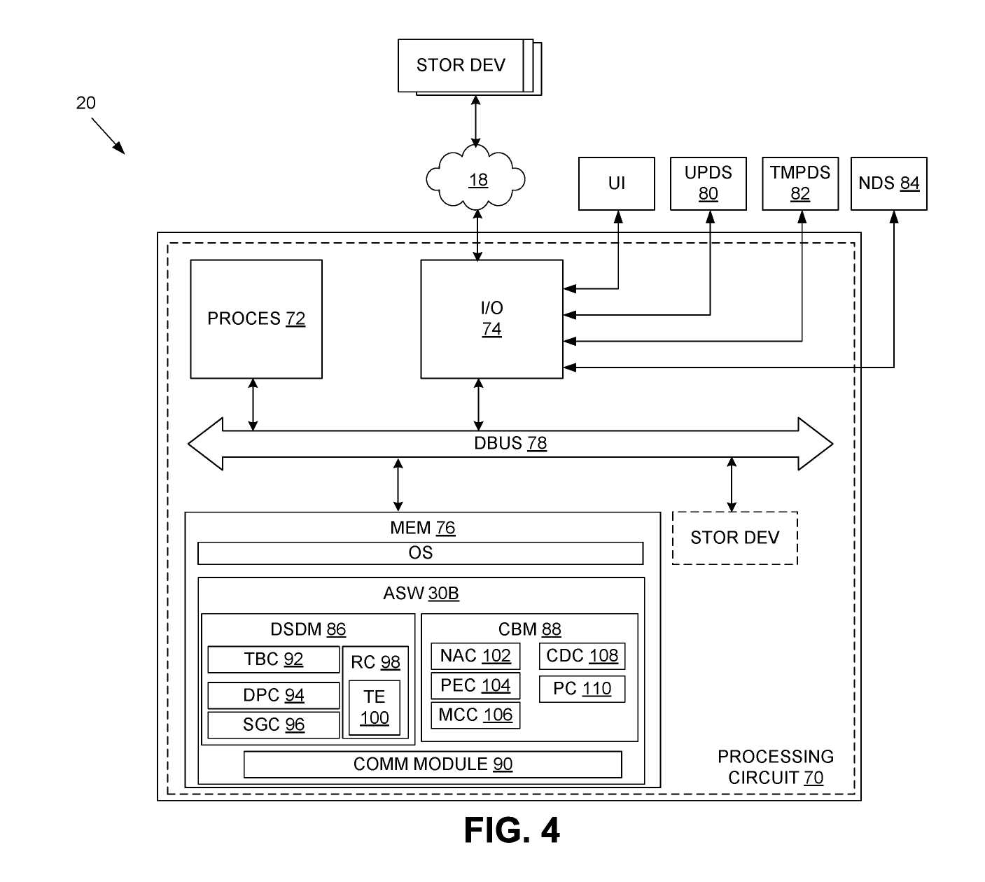

The electronics device 14 may be embodied as a smartphone, mobile phone, cellular phone, pager, stand-alone image capture device (e.g., camera), laptop, workstation, among other handheld and portable computing/communication devices, including communication devices having wireless communication capability, including telephony functionality. It is noted that if the electronics device 14 is embodied as a laptop or computer in general, the architecture more resembles that of the computing system 20 shown and described in association with FIG. 4. In some embodiments, the electronics device 14 may have built-in, image capture/recording functionality. In the depicted embodiment of FIG. 1, the electronics device 14 is a smartphone, though it should be appreciated that the electronics device 14 may take the form of other types of devices as described above. Further discussion of the electronics device 14 is described below in association with FIG. 3.

The cellular network 16 may include the necessary infrastructure to enable cellular communications by the electronics device 14 and optionally the wearable device 12. There are a number of different digital cellular technologies suitable for use in the cellular network 16, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others.

The wide area network 18 may comprise one or a plurality of networks that in whole or in part comprise the Internet. The electronics device 14 and optionally wearable device 12 access one or more of the devices of the computing system 20 via the Internet 18, which may be further enabled through access to one or more networks including PSTN (Public Switched Telephone Networks), POTS, Integrated Services Digital Network (ISDN), Ethernet, Fiber, DSL/ADSL, among others.

The computing system 20 comprises one or more devices coupled to the wide area network 18, including one or more computing devices networked together, including an application server(s) and data storage. The computing system 20 may serve as a cloud computing environment (or other server network) for the electronics device 14 and/or wearable device 12, performing processing and data storage on behalf of (or in some embodiments, in addition to) the electronics devices 14 and/or wearable device 12. In one embodiment, the computing system 20 may be configured to be a backend server for a health program. The computing system 20 receives data collected via one or more of the wearable device 12 or electronics device 14 and/or other devices or applications, stores the received data in a user profile data structure (e.g., database), and generates the notifications for presentation to the user. The computing system 20 is programmed to handle the operations of one or more health or wellness programs implemented on the wearable device 12 and/or electronics device 14 via the networks 16 and/or 18. For example, the computing system 20 processes user registration requests, user device activation requests, user information updating requests, data uploading requests, data synchronization requests, etc. The data received at the computing system 20 may be a plurality of measurements pertaining to the parameters, for example, body movements and activities, heart rate, respiration rate, blood pressure, body temperature, light and visual information, etc. and the corresponding context. Based on the data observed during a period of time and/or over a large population of users, the computing system 20 generates statements pertaining to each specific parameter, and provides the statements via the networks 16 and/or 18 as an on-going narrative of statements or notifications for presentation on devices 12 and/or 14. In some embodiments, the computing system 20 is configured to be a backend server for a health-related program or a health-related application implemented on the mobile devices. The functions of the computing system 20 described above are for illustrative purpose only. The present disclosure is not intended to be limiting. The computing system 20 may be a general computing server or a dedicated computing server. The computing system 20 may be configured to provide backend support for a program developed by a specific manufacturer. However, the computing system 20 may also be configured to be interoperable across other servers and generate statements in a format that is compatible with other programs. In some embodiments, one or more of the functionality of the computing system 20 may be performed at the respective devices 12 and/or 14. Further discussion of the computing system 20 is described below in association with FIG. 4.

An embodiment of a notification system may comprise the wearable device 12, the electronics device 14, and/or the computing system 20. In other words, one or more of the aforementioned devices 12, 14, and 20 may implement the functionality of the notification system. For instance, the wearable device 12 may comprise all of the functionality of a notification system, enabling the user to avoid the need for Internet connectivity and/or carrying a smartphone 14 around. In some embodiments, the functionality of the notification system may be implemented using a combination of the wearable device 12 and the electronics device 14 and/or the computing system 20 (with or without the electronics device 14). For instance, the wearable device 12 and/or the electronics device 14 may present notifications via a user interface and provide sensing functionality, yet rely on remote data structures of the computing system 20 and remote processing of the computing systems 20 (e.g., defining of the data structures, measure computations, adding nodes for the formation of a chain, etc.). In other words, the defining of data structures that are contextually related based on the user data (e.g., received from the devices 12, 14, databases of user data, location-determining sources, etc.) and the processing related to the data structures (e.g., labeling or associating of starting, intermediate, and target nodes, determination of plural (e.g., all) paths from starting and intermediate nodes, the computing of measures or scores based on predefined criteria, and the selection of nodes of notifications according to presentation in a narrative format) may be implemented on any one or a combination of devices 12, 14, and 20.

As an example, the wearable device 12 may monitor activity of the user, and communicate context and the sensed parameters (e.g., location coordinates, motion data, physiological data, etc.) to one of the devices (e.g., the electronics device 14 and/or the computing system 20) external to the wearable device 12, the latter where all other processing is performed, and then each notification may be generated at one of the devices remote to the wearable device 12 and communicated back to the wearable device 12 for presentation according to a given temporal order (e.g., at different time intervals) relative to the presentation of other notifications. One benefit to the latter embodiment is that off-loading of the computational resources of the wearable device 12 is enabled, conserving power consumed by the wearable device 12. In some embodiments, the notifications may be presented by the wearable device 12 and/or the electronics device 14 and all other processing may be performed by the computing system 20, and in some embodiments, the notifications may be presented by the wearable device 12 and/or the electronics device 14 and all other processing performed by the electronics device 14, and in some embodiments, the notifications and processing may be entirely performed by the wearable device 12 and/or the electronics device 14. These and/or other variations are contemplated to be within the scope of the disclosure. For instance, in some embodiments, networks and devices associated with the notification system may be configured to be the same for all users, or customized for a sub-population, including created separately for each user.

Attention is now directed to FIG. 2, which illustrates an example wearable device 12 in which all or a portion of the functionality of a notification system may be implemented. That is, FIG. 2 illustrates an example architecture (e.g., hardware and software) for the example wearable device 12. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 22 (e.g., 22A-22N), one or more signal conditioning circuits 24 (e.g., SIG COND CKT 24A-SIG COND CKT 24N) coupled respectively to the sensors 22, and a processing circuit 26 (PROCES CKT) that receives the conditioned signals from the signal conditioning circuits 24. In one embodiment, the processing circuit 26 comprises an analog-to-digital converter (ADC), a digital-to-analog converter (DAC), a microcontroller unit (MCU), a digital signal processor (DSP), and memory (MEM) 28. In some embodiments, the processing circuit 26 may comprise fewer or additional components than those depicted in FIG. 2. For instance, in one embodiment, the processing circuit 26 may consist of the microcontroller. In some embodiments, the processing circuit 26 may include the signal conditioning circuits 24. The memory 28 comprises an operating system (OS) and application software (ASW) 30. The application software 30 comprises a plurality of software modules (e.g., executable code/instructions) including sensor measurement software (SMSW) 32, communications software (CMSW) 34, and notification presentation software (NPSW) 36. In some embodiments, the application software 30 may include additional software that implements the defining of data structure, associations with nodes, establishment of predetermined pathways, and additional processing (e.g., node selection and linking for chain construction). For purposes of brevity, the description about the application software 30 hereinafter is premised on the assumption that data structure definitions and processing to provide notifications is performed at the computing system 20 (explained below in association with the computing system 20), and the presentation of those notifications is performed in different instances at the wearable device 12 after receiving the respective notifications during non-overlapping intervals based on the current context from the computing system 20 directly or via communications with the electronics device 14 (which receives the notifications from the computing system 20). The sensor measurement software 32 comprises executable code to process the signals (and associated data) measured by the sensors 22 and record and/or derive physiological parameters, such as heart rate, blood pressure, respiration, perspiration, etc. and movement and/or location data.

The communications software 34 comprises executable code/instructions to enable a communications circuit 38 of the wearable device 12 to operate according to one or more of a plurality of different communication technologies (e.g., NFC, Bluetooth, Wi-Fi, including 802.11, GSM, LTE, CDMA, WCDMA, Zigbee, etc.). The communications software 34 instructs and/or controls the communications circuit 38 to transmit the raw sensor data and/or the derived information from the sensor data to the computing system 20 (e.g., directly via the cellular network 16, or indirectly via the electronics device 14). The communications software 34 may also include browser software in some embodiments to enable Internet connectivity. The communications software 34 may also be used to access certain services, such as mapping/place location services, which may be used to determine context for the sensor data. These services may be used in some embodiments of a notification system, and in some instances, may not be used. In some embodiments, the communications software 34 may be external to the application software 30 or in other segments of memory. The notification presentation software 36 is configured to receive the notifications via the communications software 34 and communications circuit 38 as the notifications are communicated at different (non-overlapping) intervals based on the context (e.g., determined by the computing system 20 from the input data received from the wearable device 12). The notification presentation software 36 may format and present the notifications at an output interface 40 of the wearable device 12 at a time corresponding to when the notifications are received from the computing system 20 and/or electronics device 14 and/or at other times during the day or evening if different than when received. In some embodiments, the notification presentation software 36 may learn (e.g., based on previous notifications that were indicated, such as via feedback or use or neglect of similar and/or previous notifications) a preferred or best moment to present a current notification received from the computing system 20.

As indicated above, in one embodiment, the processing circuit 26 is coupled to the communications circuit 38. The communications circuit 38 serves to enable wireless communications between the wearable device 12 and other devices, including the electronics device 14 and the computing system 20, among other devices. The communications circuit 38 is depicted as a Bluetooth circuit, though not limited to this transceiver configuration. For instance, in some embodiments, the communications circuit 38 may be embodied as any one or a combination of an NFC circuit, Wi-Fi circuit, transceiver circuitry based on Zigbee, 802.11, GSM, LTE, CDMA, WCDMA, among others such as optical or ultrasonic based technologies. The processing circuit 26 is further coupled to input/output (I/O) devices or peripherals, including an input interface 42 (INPUT) and the output interface 40 (OUT). Note that in some embodiments, functionality for one or more of the aforementioned circuits and/or software may be combined into fewer components/modules, or in some embodiments, further distributed among additional components/modules or devices. For instance, the processing circuit 26 may be packaged as an integrated circuit that includes the microcontroller (microcontroller unit or MCU), the DSP, and memory 28, whereas the ADC and DAC may be packaged as a separate integrated circuit coupled to the processing circuit 26. In some embodiments, one or more of the functionality for the above-listed components may be combined, such as functionality of the DSP performed by the microcontroller.

The sensors 22 are selected to perform detection and measurement of a plurality of physiological and behavioral parameters (e.g., typical behavioral parameters or activities including walking, running, cycling, and/or other activities, including shopping, walking a dog, working in the garden, etc.), including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body mass index or BMI). At least one of the sensors 22 may be embodied as movement detecting sensors, including inertial sensors (e.g., gyroscopes, single or multi-axis accelerometers, such as those using piezoelectric, piezoresistive or capacitive technology in a microelectromechanical system (MEMS) infrastructure for sensing movement) and/or as GNSS sensors, including a GPS receiver to facilitate determinations of distance, speed, acceleration, location, altitude, etc. (e.g., location data, or generally, sensing movement), in addition to or in lieu of the accelerometer/gyroscope and/or indoor tracking (e.g., ibeacons, WiFi, coded-light based technology, etc.). The sensors 22 may also include flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. The sensors 22 may include other and/or additional types of sensors for the detection of, for instance, barometric pressure, humidity, outdoor temperature, etc. In some embodiments, GNSS functionality may be achieved via the communications circuit 38 or other circuits coupled to the processing circuit 26.

The signal conditioning circuits 24 include amplifiers and filters, among other signal conditioning components, to condition the sensed signals including data corresponding to the sensed physiological parameters and/or location signals before further processing is implemented at the processing circuit 26. Though depicted in FIG. 2 as respectively associated with each sensor 22, in some embodiments, fewer signal conditioning circuits 24 may be used (e.g., shared for more than one sensor 22). In some embodiments, the signal conditioning circuits 24 (or functionality thereof) may be incorporated elsewhere, such as in the circuitry of the respective sensors 22 or in the processing circuit 26 (or in components residing therein). Further, although described above as involving unidirectional signal flow (e.g., from the sensor 22 to the signal conditioning circuit 24), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 22, with the sensor 22 (e.g., photocell) receiving the reflected/refracted signals.

The communications circuit 38 is managed and controlled by the processing circuit 26 (e.g., executing the communications software 34). The communications circuit 38 is used to wirelessly interface with the electronics device 14 (FIG. 3) and/or one or more devices of the computing system 20. In one embodiment, the communications circuit 38 may be configured as a Bluetooth transceiver, though in some embodiments, other and/or additional technologies may be used, such as Wi-Fi, GSM, LTE, CDMA and its derivatives, Zigbee, NFC, among others. In the embodiment depicted in FIG. 2, the communications circuit 38 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 26. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 38 may be implemented by the microcontroller, the DSP, or a combination of both. In some embodiments, the communications circuit 38 may have its own dedicated controller that is supervised and/or managed by the microcontroller.

In one example operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 26) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 28 stores the communications software 34, which when executed by the microcontroller, controls the Bluetooth (and/or other protocols) transmission/reception.

Though the communications circuit 38 is depicted as an IF-type transceiver, in some embodiments, a direct conversion architecture may be implemented. As noted above, the communications circuit 38 may be embodied according to other and/or additional transceiver technologies.

The processing circuit 26 is depicted in FIG. 2 as including the ADC and DAC. For sensing functionality, the ADC converts the conditioned signal from the signal conditioning circuit 24 and digitizes the signal for further processing by the microcontroller and/or DSP. The ADC may also be used to convert analogs inputs that are received via the input interface 42 to a digital format for further processing by the microcontroller. The ADC may also be used in baseband processing of signals received via the communications circuit 38. The DAC converts digital information to analog information. Its role for sensing functionality may be to control the emission of signals, such as optical signals or acoustic signal, from the sensors 22. The DAC may further be used to cause the output of analog signals from the output interface 40. Also, the DAC may be used to convert the digital information and/or instructions from the microcontroller and/or DSP to analog signal that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, with comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 12. For instance, encoding functionality may involve encoding commands or data corresponding to transfer of information to the electronics device 14 or a device of the computing system 20. Also, decoding functionality may involve decoding the information received from the sensors 22 (e.g., after processing by the ADC).

The microcontroller comprises a hardware device for executing software/firmware, particularly that stored in memory 28. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters or location coordinates based on the sensors 22, and for enabling communication with the electronics device 14 and/or a device of the computing system 20, and for the presentation of a chain of notifications for the notification system.

The memory 28 can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 28 may incorporate electronic, magnetic, and/or other types of storage media.

The software in memory 28 may include one or more separate programs, each of which comprises an ordered listing of executable instructions for implementing logical functions. In the example of FIG. 2, the software in the memory 28 includes a suitable operating system and the application software 30, which includes a plurality of software modules 32-36 for implementing certain embodiments of a notification system and algorithms for determining physiological and/or behavioral measures and/or other information (e.g., including location, speed of travel, etc.) based on the output from the sensors 22. The raw data from the sensors 22 may be used by the algorithms to determine various physiological and/or behavioral measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14 or one or more devices of the computing system 20) in lieu of, or in addition to, the computations performed local to the wearable device 12. In some embodiments, the GPS functionality of the sensors 22 collects contextual data (time and location data, including location coordinates). The application software 30 may collect location data by sampling the location readings from the sensor 22 over a period of time (e.g., minutes, hours, days, weeks, etc.). The application software 30 may also collect information about the means of ambulation. For instance, the GPS data (which may include time coordinates) may be used by the application software 30 to determine speed of travel, which may indicate whether the user is moving within a vehicle, on a bicycle, or walking or running. In some embodiments, other and/or additional data may be used to assess the type of activity, including physiological data (e.g., heart rate, respiration rate, galvanic skin response, etc.) and/or behavioral data.

The operating system essentially controls the execution of other computer programs, such as the application software 30 and associated modules 32-36, and provides scheduling, input-output control, file and data management, memory management, and communication control and related services. The memory 28 may also include user data, including weight, height, age, gender, goals, body mass index (BMI) that are used by the microcontroller executing the executable code of the algorithms to accurately interpret the measured physiological and/or behavioral data. The user data may also include historical data relating past recorded data to prior contexts.

Although the application software 30 (and component parts 32-36) are described above as implemented in the wearable device 12, some embodiments may distribute the corresponding functionality among the wearable device 12 and other devices (e.g., electronics device 14 and/or one or more devices of the computing system 20), or in some embodiments, the application software 30 (and component parts 32-36) may be implemented in another device (e.g., the electronics device 14).

The software in memory 28 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 42 comprises an interface (e.g., including a user interface) for entry of user input, such as a button or microphone or sensor (e.g., to detect user input) or touch-type display. In some embodiments, the input interface 42 may serve as a communications port for downloaded information to the wearable device 12 (such as via a wired connection). The output interfaces 40 comprises an interface for the presentation or transfer of data, including a user interface (e.g., display screen presenting a graphical user interface) or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). For instance, the output interface 40 may be used to present the notifications to the user. In some embodiments, at least some of the functionality of the input and output interfaces 42 and 40, respectively, may be combined, including being embodied at least in part as a touch-type display screen for the entry of input (e.g., to select an opportunity for behavioral change, such as via a presented invite in a dashboard or other screen, to input preferences, etc.) and presentation of notifications, among other data. In some embodiments, selection may be made automatically after the invitation based on detecting the context of the user (e.g., a context aware feature).

Referring now to FIG. 3, shown is an example electronics device 14 in which all or a portion of the functionality of a notification system may be implemented. Similar to the description for the wearable device 12 of FIG. 2, and for the sake of brevity, the application software of the electronics device 14 comprises similar components as that for the wearable device 12, with the understanding that fewer or a greater number of software modules of the notification system may be used in some embodiments. In the depicted example, the electronics device 14 is embodied as a smartphone (hereinafter, referred to smartphone 14), though in some embodiments, other types of devices may be used, such as a workstation, laptop, notebook, tablet, etc. It should be appreciated by one having ordinary skill in the art that the logical block diagram depicted in FIG. 3 and described below is one example, and that other designs may be used in some embodiments. The application software 30A comprises a plurality of software modules (e.g., executable code/instructions) including sensor measurement software (SMSW) 32A and notification presentation software (NPSW) 36A, as well as communications software as is expected of mobile phones. In some embodiments, the application software 30A may include additional software that implements data structure definitions, associations, and additional processing. Note that the application software 30A (and component parts 32A and 36A) comprise at least some of the functionality of the application software 30 (and component parts 32 and 36) described above for the wearable device 12, and may include additional software pertinent to smartphone operations (e.g., possibly not found in wearable devices 12). The smartphone 14 comprises at least two different processors, including a baseband processor (BBP) 44 and an application processor (APP) 46. As is known, the baseband processor 44 primarily handles baseband communication-related tasks and the application processor 46 generally handles inputs and outputs and all applications other than those directly related to baseband processing. The baseband processor 44 comprises a dedicated processor for deploying functionality associated with a protocol stack (PROT STK) 48, such as a GSM (Global System for Mobile communications) protocol stack, among other functions. The application processor 46 comprises a multi-core processor for running applications, including all or a portion of the application software 30A and its corresponding component parts 32A and 36A as described above in association with the wearable device 12 of FIG. 2. The baseband processor 44 and application processor 46 have respective associated memory (e.g., MEM) 50, 52, including random access memory (RAM), Flash memory, etc., and peripherals, and a running clock.

More particularly, the baseband processor 44 may deploy functionality of the protocol stack 48 to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor 44 manages radio communications and control functions, including signal modulation, radio frequency shifting, and encoding. The baseband processor 44 comprises, or may be coupled to, a radio (e.g., RF front end) 54 and/or a GSM modem having one or more antennas, and analog and digital baseband circuitry (ABB, DBB, respectively in FIG. 3). The radio 54 comprises a transceiver and a power amplifier to enable the receiving and transmitting of signals of a plurality of different frequencies, enabling access to the cellular network 16 (FIG. 1), and hence the communication of user data and associated contexts to the computing system 20 (FIG. 1) and the receipt of notifications from the computing system 20. The analog baseband circuitry is coupled to the radio 54 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 46 or directly from the smartphone user interface 56 (e.g., microphone, earpiece, ring tone, vibrator circuits, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 46). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides to the analog baseband circuitry (e.g., converted to analog signals by the DAC).

The application processor 46 operates under control of an operating system (OS) that enables the implementation of a plurality of user applications, including the application software 30A. The application processor 46 may be embodied as a System on a Chip (SOC), and supports a plurality of multimedia related features including web browsing to access one or more computing devices of the computing system 20 (FIG. 4) that are coupled to the Internet, email, multimedia entertainment, games, etc. For instance, the application processor 46 may execute interface software (e.g., middleware, such as a browser with or operable in association with one or more application program interfaces (APIs)) to enable access to a cloud computing framework or other networks to provide remote data access/storage/processing, and through cooperation with an embedded operating system, access to calendars, location services, reminders, etc. For instance, in some embodiments, the notification system may operate using cloud computing, where the processing of sensor data (e.g., location data, including data received from the wearable device 12 or from integrated sensors within the smartphone 14, including motion sense, image capture, location detect, etc.) and context may be achieved by one or more devices of the computing system 20. The application processor 46 generally comprises a processor core (Advanced RISC Machine or ARM), and further comprises or may be coupled to multimedia modules (for decoding/encoding pictures, video, and/or audio), a graphics processing unit (GPU), communication interfaces (COMM) 58, and device interfaces. The communication interfaces 58 may include wireless interfaces, including a Bluetooth (BT) (and/or Zigbee in some embodiments) module that enables wireless communication with an electronics device, including the wearable device 12, other electronics devices, and a Wi-Fi module for interfacing with a local 802.11 network. The application processor 46 further comprises, or is coupled to, a global navigation satellite systems (GNSS) transceiver or receiver (GNSS) 60 for access to a satellite network to, for instance, provide location services. The device interfaces coupled to the application processor 46 may include the user interface 56, including a display screen. The display screen, similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the display screen may be used to present web pages, dashboards, notifications, and/or other documents or data received from the computing system 20 and/or the display screen may be used to present information (e.g., notifications) in graphical user interfaces (GUIs) rendered locally in association with the application software 30A. Other user interfaces 56 include a keypad, microphone, speaker, ear piece connector, I/O interfaces (e.g., USB (Universal Serial Bus)), SD/MMC card, among other peripherals. Also coupled to the application processor 46 is an image capture device (IMAGE CAPTURE) 62. The image capture device 62 comprises an optical sensor (e.g., a charged coupled device (CCD) or a complementary metal-oxide semiconductor (CMOS) optical sensor). The image capture device 62 may be used to detect various physiological parameters of a user, including blood pressure based on remote photoplethysmography (PPG). Also included is a power management device 64 that controls and manages operations of a battery 66. The components described above and/or depicted in FIG. 3 share data over one or more busses, and in the depicted example, via data bus 68. It should be appreciated by one having ordinary skill in the art, in the context of the present disclosure, that variations to the above may be deployed in some embodiments to achieve similar functionality.

In the depicted embodiment, the application processor 46 runs the application software 30A, which in one embodiment, includes a plurality of software modules (e.g., executable code/instructions) including the sensor measurement software (SMSW) 32A and the notification presentation software (NPSW) 36A. Since the description of the application software 30 and software modules 32 and 36 has been described above in association with the wearable device 12 (FIG. 2), and since the same functionality is present in software 32A (albeit on perhaps different sensor data) and 36A, discussion of the same here is omitted for brevity. It is noteworthy, however, that some or all of the software functionality may be implemented in the smartphone 14. For instance, all of the functionality of the application software 30A may be implemented in the smartphone 14, or functionality of the application software 30A may be divided among plural devices of the environment 10 (FIG. 1) in some embodiments. The application software 30A may also comprises executable code to process the signals (and associated data) measured by the sensors (of the wearable device 12 as communicated to the smartphone 14, or based on sensors integrated within the smartphone 14) and record and/or derive physiological parameters, such as heart rate, blood pressure, respiration, perspiration, etc. Note that functionality of the software modules 32A and 36A, similar to those described for the wearable device 12, may be combined in some embodiments, or further distributed among additional modules. In some embodiments, the execution of the application software 30A and associated modules 32A and 36A may be distributed among plural devices, as set forth above. Note that all or a portion of the aforementioned hardware and/or software of the smartphone 14 may be referred to herein as a processing circuit.

Referring now to FIG. 4, shown is a computing system 20 in which at least a portion of the functionality of a notification system may be implemented. The computing system 20 may comprise a single computing device as shown here, or in some embodiments, may comprise plural devices that collectively perform the functionality described below. In one embodiment, the computing system 20 may be embodied as an application server, a computer, among other computing devices. One having ordinary skill in the art should appreciate in the context of the present disclosure that the example computing system 20 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. The computing system 20 is depicted in this example as a computer system, including one providing a function of an application server. It should be appreciated that certain well-known components of computer systems are omitted here to avoid obfuscating relevant features of the computing system 20. In one embodiment, the computing system 20 comprises a processing circuit 70 comprising hardware and software components. In some embodiments, the processing circuit 70 may comprise additional components or fewer components. For instance, memory may be separate. The processing circuit 70 comprises one or more processors, such as processor 72 (PROCES), input/output (I/O) interface(s) 74 (I/O), and memory 76 (MEM), all coupled to one or more data busses, such as data bus 78 (DBUS). The memory 76 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 76 may store a native operating system (OS), one or more native applications, emulation systems, or emulated applications for any of a variety of operating systems and/or emulated hardware platforms, emulated operating systems, etc. In some embodiments, the processing circuit 70 may include, or be coupled to, one or more separate storage devices. For instance, in the depicted embodiment, the processing circuit 70 is coupled via the I/O interfaces 74 to user profile data structures (UPDS) 80, template data structures (TMPDS) 82, and notification data structures (NDS) 84, explained further below. In some embodiments, the user profile data structures 80, the template data structures 82, and the notifications data structures 84 may be coupled to the processing circuit 70 via the data bus 78 or coupled to the processing circuit 70 via the I/O interfaces 74 as network-connected storage devices (STOR DEVS). The data structures 80, 82, and 84 may be stored in persistent memory (e.g., optical, magnetic, and/or semiconductor memory and associated drives). In some embodiments, the data structures 80, 82, and 84 may be stored in memory 76. The user profile data structures 80 are configured to store user profile data including the real-time measurements of the parameters for a large population of users, personal information of the large population of users, previously generated statements related to the large population of users, etc. In some embodiments, the user profile data structures 80 are configured to store health-related information of the user. The user profile data is organized to model various aspects of a user in a way that supports simple querying as well as complicated data analysis. The user profile data structures 80 may be a backend database of the computing system 20. In some embodiments, however, the user profile data structures 80 may be in the form of network storage and/or cloud storage directly connected to the network 18. In some embodiments, the user profile data structures 80 may serve as backend storage of the computing system 20 as well as network storage and/or cloud storage. The user profile data structures 80 are updated periodically, aperiodically, and/or in response to a request from the wearable device 12, the electronics device 14, and/or the operations of the computing system 20. The template data structures 82 are configured to store one or more templates that are used in the statement definition stage to generate the statements conveying information to the user. The statements for different objectives may use different templates. For example, education related statements may apply templates with referral links to educational resources; feedback on performance may apply templates with rating/ranking comments, etc.

The template data structures 82 may be maintained by an administrator operating the computing system 20. The template data structures 82 may be updated based on the usage of each template, the feedback on each generated statement, etc. The templates that are more often used and/or receive more positive feedbacks from the users may be highly recommended to generate the statements in the future. In some embodiments, the templates may be general templates that can be used to generate all types of statements. In some other embodiments, the templates may be classified into categories, each category pertaining to a parameter. For example, templates for generating statement pertaining to heart rate may be partially different from templates for generating statement pertaining to sleep quality.

The notifications data structures 84 are configured to store the statements that are constructed based on the templates and used in the design phase of the chaining of notifications. In the embodiment depicted in FIG. 4, the memory 76 comprises an operating system (OS) and the application software 30B. The application software 30B comprises a data structures definition module (DSDM) 86, a chain building module (CBM) 88, and a communications module 90. The data structures definition module 86 comprises a plurality of sub-modules, including a template building component (TBC) 92, a data processing component (DPC) 94, a statement generating component (SGC) 96, and a ranking component (RC) 98 (which includes a truth engine (TE) 100). The chain building module 88 comprises a plurality of sub-modules, including a node association component (NAC) 102, a pathway establishment component (PEC) 104, a measures compute component (MCC) 106, a chain determining component (CDC) 108, and a presentation component (PC) 110. In one embodiment, the chain building module 88 comprises a design component (e.g., node association component 102 and pathway establishment component 104) that is implemented by a content author interacting with the user interface of the computing system 20, and an execution component implemented algorithmically via the measures compute component 106, chain determining component 108, and the presentation component 110. In some embodiments, functionality for the design components may be implemented in the execution stage. For instance, nodes may be updated or changed (including their connections) during execution in some embodiments.

Referring to the components of the data structures definition module 86, each of the one or more computer programmed components comprises a set of algorithms implemented on the processor 72 that instructs the processor 72 to perform one or more functions related to generating the statements, and/or other operations. For example, the template building component 92 comprises algorithms implemented on the processor 72 that instruct the processor 72 to build one or more templates for generating the statements; the data processing component 94 comprises algorithms implemented on the processor 72 that instruct the processor 72 to analyze the received data received via the I/O interfaces 74; the statement generating component 96 comprises algorithms implemented on the processor 72 that instruct the processor 72 to generate one or more statements pertaining to a parameter. In some embodiments, the statements may be ranked for each parameter by the ranking component 98 implementing a truth engine 100. In some embodiments, ranking may not be implemented. The statements are stored in the notifications data structures 84 or in other storage and accessed by a content author for use in conjunction with the chain building module 88.

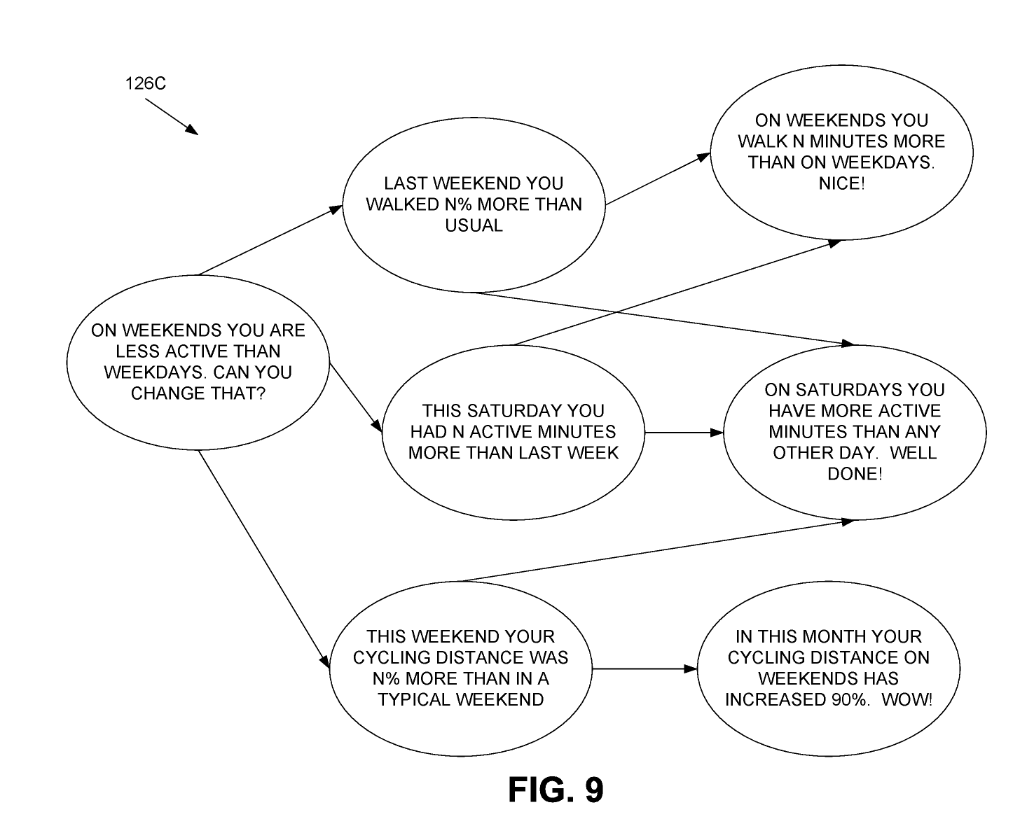

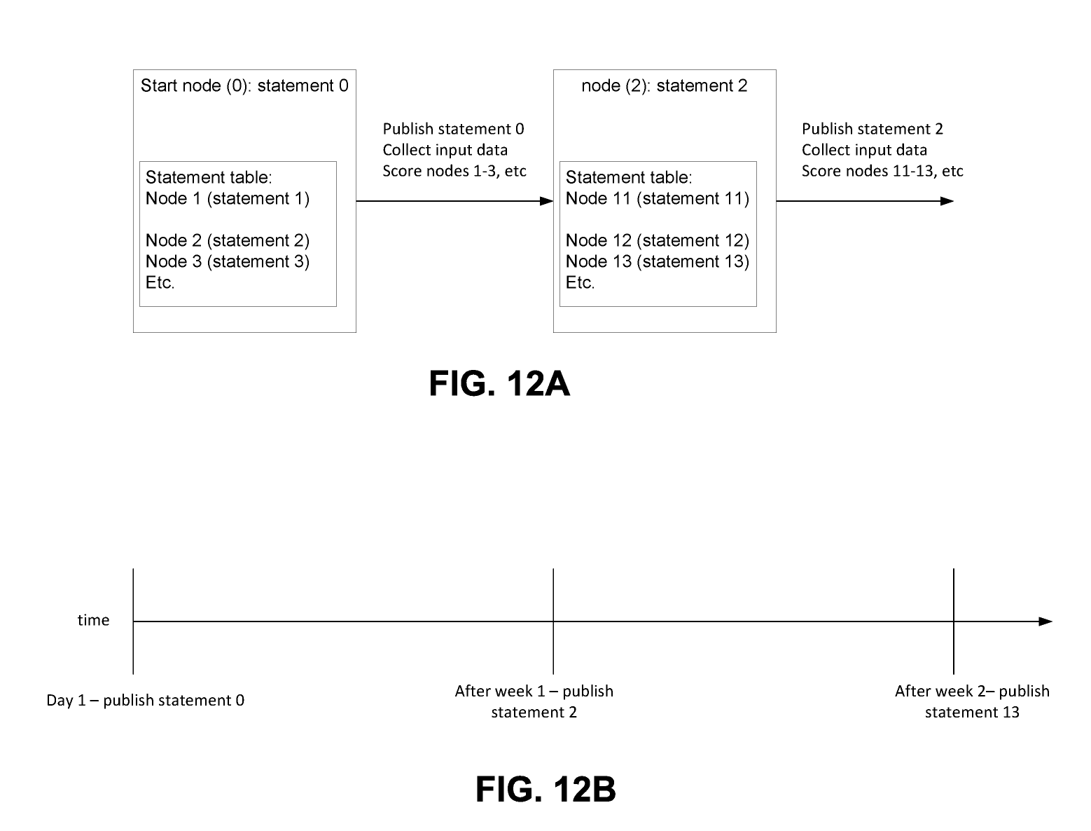

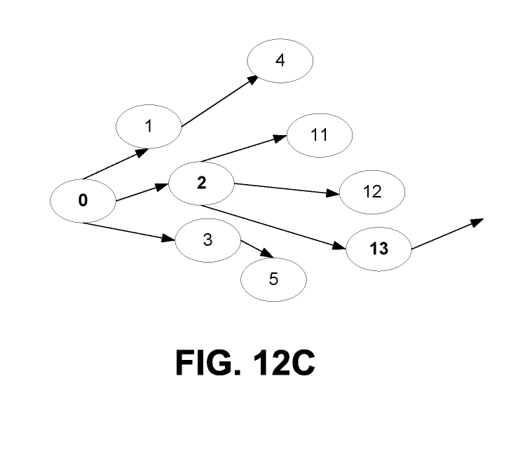

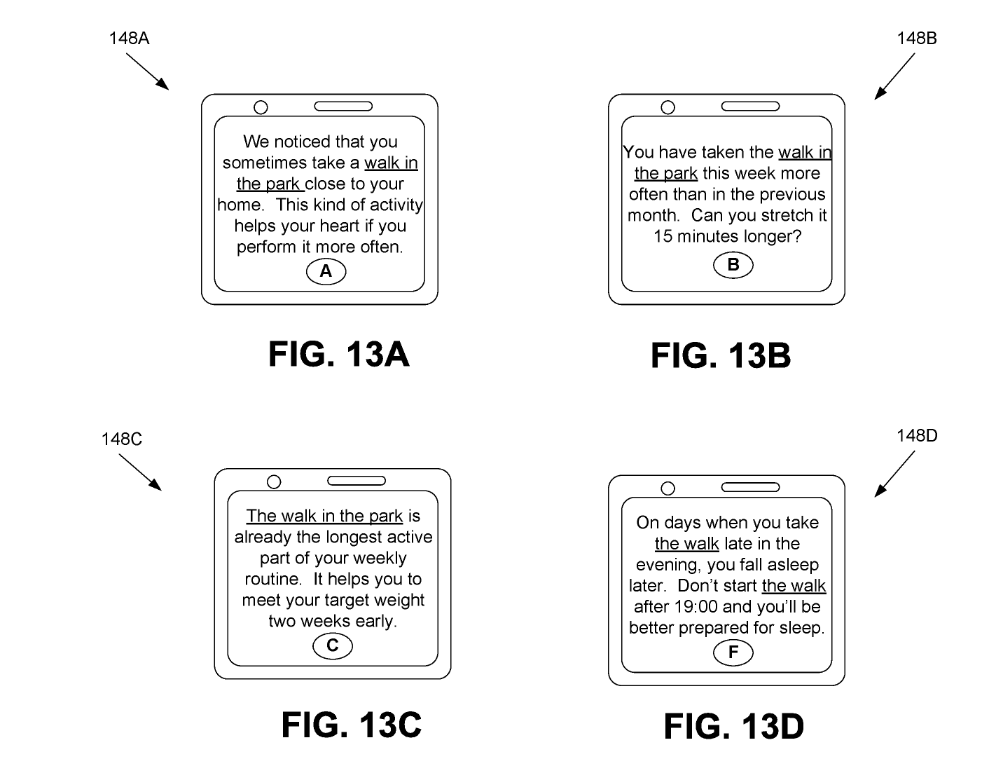

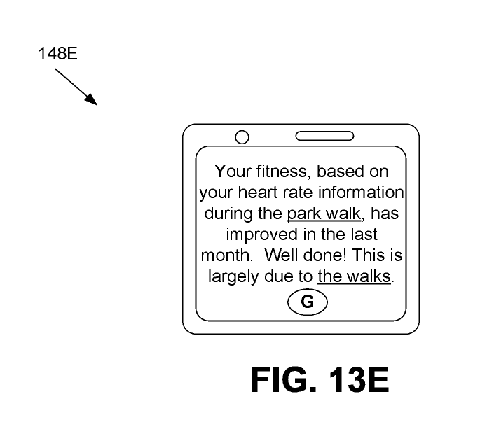

Explaining the components of the chain building module 88 further, the node association component 102 is used by the content author to associate the predetermined data structures (e.g., notifications or statements) provided by the data structure definition module 86 (and stored in the notifications data structures 84) with either starting nodes, intermediate nodes, or target nodes. For instance, the content author may be presented via the node association component 102 a graphical user interface (or other interface of a software tool) that enables the content author to label the statements based on their content as start- or end-points (e.g., starting nodes and target nodes, respectively) of narratives. In one embodiment, start-point content refers to an opportunity, e.g., “On Saturdays after work you are typically less active than other evenings. Can you consider becoming more active then?”, and an end-point could be, for example, a positive observation related to a measure “Your average walking distance in Saturday evenings is 50% more than other work days. Well done! This really helps you to reach your targets earlier.” The content author further labels the statements he or she deems as appropriate as intermediate statements. The intermediate nodes comprise intermediate data structures (e.g., notifications or statements) that comprise a network of all possible intermediate statements that join the start points with the end points. Collectively, the plurality of intermediate nodes comprises a network of intermediate nodes. The pathway establishment component 104 is another software tool used by the content author to establish plural (e.g., all) possible pathways from the starting nodes to the target nodes (e.g., end points). A path or pathway refers to a narrative, which is a chain of statements that leads from a start-point (e.g., starting node) to an end-point (e.g., target node). Using the pathway establishment component 104 (e.g., another interactive GUI), the content author identifies all possible paths in the collection of statements, and populates a statement table for each of the starting and ending nodes based on the possible paths. In other words, each starting and ending node has a statement table that lists all possible next nodes (next statements) along with plural parameters for each statement (e.g., as described further in association with FIG. 12). In one embodiment, there are typically several possible paths from a start-point to an end-point and there are also multiple paths to different end-points from one start-point. An intermediate statement in the path example introduced above could be for example, “This Saturday you have been more active than on typical Saturday evenings”. An example of two types of paths or pathways is illustrated in FIGS. 9A and 9B, and a further example of operations of the chain building module 88 is described in association with FIGS. 13A-13C, described further below. The chain building module 88 receives input data over a certain interval of time. The interval of time may be user configured or pre-programmed. During the monitoring of a user (e.g., receiving input data from the wearable device 12 and/or the electronics device 14) and/or at predetermined intervals or after certain events (e.g., after determining an opportunity for positive behavioral change), the measures compute component 106 computes measures or scores for the starting point statements (starting nodes)6 based on the input data.

The chain determining component 108 selects one of the starting statements to commence chain building. Selection of one of the statements is based on one of the starting statements meeting the highest computed score and also meeting a predetermined criteria (e.g., at or above a threshold value). The chain determining component 108 provides the selected statement to the presentation component 110. The presentation component 110 formats the statement as needed for delivery to the wearable device 12 and/or the electronics device 14 and provides the selected first statement as a notification to the device(s) 12, 14. The chain building module 88 continues to monitor the input data for an interval of time (e.g., one week), and the measures compute component 106 computes the scores of the statement table for the now-published starting node. The chain determining component 108 selects the highest score (meeting a predetermined criteria) among the plurality of statements (for nodes among possible pathways from the starting node) from the statement table. The selected statement may correspond to one of the intermediate nodes or an ending or target node. The presentation component 110 formats the selected statement as needed and delivers to the wearable device 12 and/or electronics device 14, and if the selected statement was not a target node, the process of receiving further input data and computing the scores for the statements of the statement table of the last published statement and publication of the next statement continues until one of the target nodes is reached or the chain building terminates (e.g., reach a termination event). In effect, the chain determining component 108 operates in conjunction with the measures compute component 106 using a chain control algorithm (described below) that, once the starting node has been identified and selected (e.g., triggering chain operations), the chain determining component 108 starts tracking the scoring of the node statements (of the statement table) in the paths. When one of the nodes scores higher than others, and above a certain threshold, the chain determining component 108 passes that statement to the presentation component 110, which communicates the statement to the electronics device 14 and/or wearable device 12 for presentation as a notification to the user. This process occurs over time until the target node has been reached (or a termination event occurs), with the result that the published nodes comprise a narrative to facilitate the progress of a user in reaching his or her goal. In other words, the chain determining component 108 (e.g., the underlying algorithm) moves to the next node, publishes, and in cooperation with the measures compute components 106 starts tracking the scores of the subsequent statements listed in the statements table for the published node. In one embodiment, the chain determining component 108 may terminate the chain process when the path tracking ends in one of the target nodes. In some embodiments, the path tracking may also be terminated, for example, if the path tracking is not proceeding from one node (e.g., within one month or some other predetermined time), the latter condition referred to herein also as a termination event.

The presentation may be presented in any one or combination of human-perceivable format (e.g., visually, audibly, using tactile feedback, including Braille, etc.). In one embodiment, the presentation component 110 may comprise card presentation functionality. As used herein, content cards generated for a specific parameter define a “family” of statements associated with the specific parameter. For example, the content cards generated for sleep quality define a family of statements related to sleep quality, while the content cards generated for running define a family of statements related to running. The content cards may be configured to present one statement per card, though in some embodiments, additional statements may be presented. Different families may define different numbers of statements for presentation. In some embodiments, the content cards may be configured to present respective statements related to the feedback of an activity performance. In some embodiments, the content cards may be configured to present statements comprising educational information. In some embodiments, the content cards may be configured to present respective statements comprising insightful analysis of the user's health-related conditions. In some embodiments, the content cards may comprise only text statements. In some embodiments, the content cards may comprise content in multiple formats including but not limited to text, audio, video, flash, hyperlink to other sources, etc. It should be appreciated that the content cards may be generated for purposes other than the examples described above, and the format of the content cards may be adjustable for presentation on different user devices. The examples set forth above are for illustrative purposes; and the present disclosure is not intended to be limiting. For instance, presentation of the notifications is not limited to content card formats.

In one embodiment, the presentation component 110 is configured to receive the statements associated with each node and configure into content card format and present the respective content cards to the user. The presentation component 110 may prepare the presentation of the content cards based on the settings pre-defined by the user and/or the configuration of each individual user device. The settings pre-defined by the user may comprise how the user wants to be notified with the content cards, for example, in a text format, in a chart format, in an audio format with low-tone female voice, in a video/flash format, and/or the combinations thereof. The settings pre-defined by the user may further comprise when and how often the user wants to be notified with the content cards, for example, every evening around 9:00 pm, every afternoon after exercise, every week, every month, in real-time, and/or the combination thereof. The settings pre-defined by the user may further comprise a preferred user device to receive the content card if the user has multiple devices. The configuration of each individual user device may include the size and resolution of the display screen of a user device, the caching space of the user device, etc. In some embodiments, the presentation component 110 may determine the connection status of the user device before sending the content cards. If the user device is determined unavailable due to power off, offline, damaged, etc., the presentation component 110 may store the generated content card in memory 76 and/or upload the generated content card to the user profile data structure 80. Once the user is detected logged-in using one of his or her user devices, the generated content card is transmitted to the user device for presentation. In some embodiments, if the preferred user device is unavailable, the presentation component 110 adjusts the content card for presentation in the logged-in user device.

In some embodiments, the presentation component 110 may convert a statement to one or more variations of the statement so that the converted statement matches a desired tone of voice, target population, or language, etc. The variations of a word and/or a statement may be acquired from a linguistic knowledge base. For example, the statement “Your sleep quality is highest after Mondays” may be converted to “You sleep well after Mondays.”

In some embodiments, the presentation component 110 may generate a large number of visual representations of a human body. The measurement data based on body sensors may be used to determine one or more images. The one or more images are further included in the content card or cards for presentation. Therefore, each content card presents a health picture of the individual, which can also be forwarded to a caregiver for reference. In some embodiments, the content card or cards may be presented in an orchestral arrangement of a melody played back to the user. It should be appreciated that the examples of card presentation described above are for illustrative purpose. The present disclosure is not intended to be limiting. In some embodiments, the presentation component 110 may supplement additional information to the statements for presentation of the content card. The additional information comprises professional advices on how to improve the user's health condition, feedbacks from a community environment, educational resources, etc.

The communications module (comm mod) 90 enables communications among network-connected devices and provides web and/or cloud services, among other software such as via one or more APIs. For instance, the communications module 90 may receive (via I/O interfaces 74) input data (e.g., a content feed) from the wearable device 12 and/or the electronics device 14 that includes sensed data and a context for the sensed data, data from third-party databases (e.g., medical data base), data from social media, data from questionnaires, data from external devices (e.g., weight scales, environmental sensors, etc.), among other data. The content feed may be continual, intermittent, and/or scheduled. The presentation component 110 operates in conjunction with the communications module 90 and the I/O interfaces 74 to provide the chain of statements or notifications to the wearable device 12 and/or the electronics device 14.

Execution of the application software 30B (and associated modules 86-90 and sub-modules 92-100 and 102-110) may be implemented by the processor 72 under the management and/or control of the operating system. The processor 72 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing system 20.

The I/O interfaces 74 comprise hardware and/or software to provide one or more interfaces to the Internet 18, as well as to other devices such as a user interface (UI) (e.g., keyboard, mouse, microphone, display screen, etc.) and/or the data structures 80-84. The user interfaces may include a keyboard, mouse, microphone, immersive head set, display screen, etc., which enable input and/or output by an administrator or other user. The I/O interfaces 74 may comprise any number of interfaces for the input and output of signals (e.g., analog or digital data) for conveyance of information (e.g., data) over various networks and according to various protocols and/or standards. The user interface (UI) is configured to provide an interface between an administrator or content author and the computing system 20. The administrator may input a request via the user interface, for instance, to manage the template database 82. Upon receiving the request, the processor 72 instructs the template building component 92 to process the request and provide information to enable the administrator to create, modify, and/or delete the templates. As indicated above, the content author may use the user interface to label statements and establish plural possible pathways among the starting, intermediate, and target nodes.

When certain embodiments of the computing system 20 are implemented at least in part with software (including firmware), as depicted in FIG. 4, it should be noted that the software (e.g., including the application software 30B (and associated modules 86-90 and sub-modules 92-100 and 102-110)) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing system 20 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

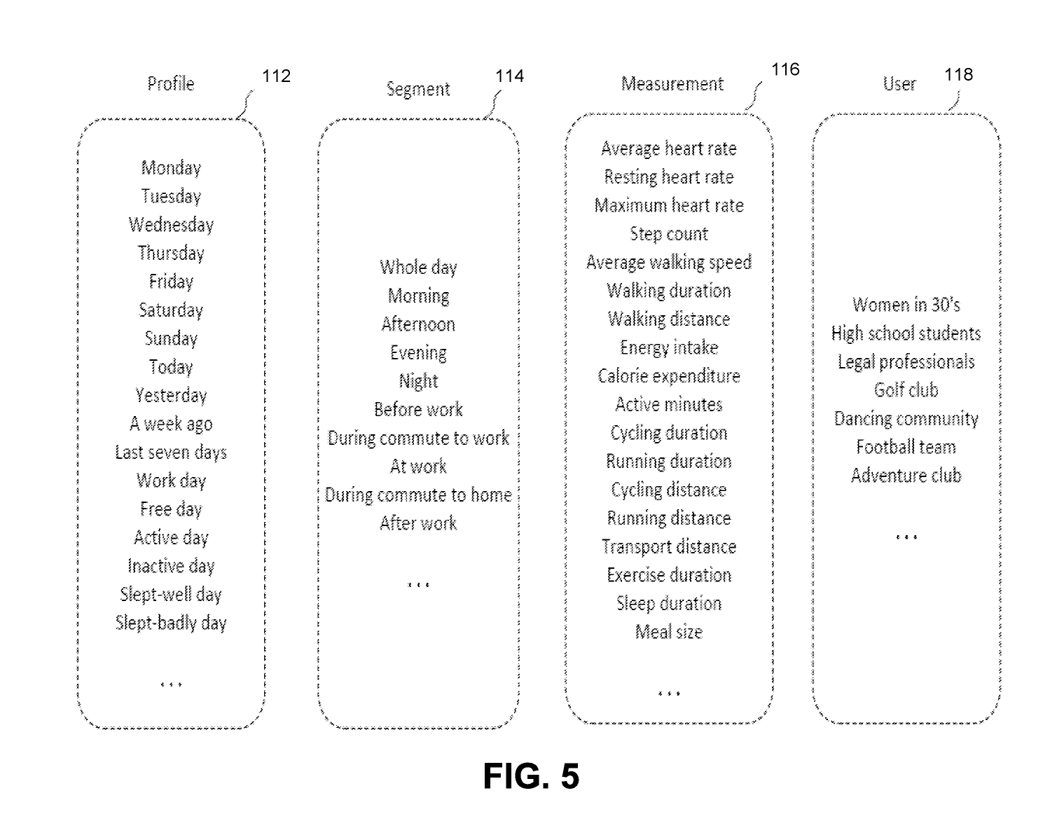

Having described the underlying hardware and software of the notification system components, attention is directed to FIGS. 5-8, which illustrates the data structure definitions functionality (e.g., statement creation) implemented by the data structures definition module 86 as implemented on the processor 72. Note that in some embodiments, functionality of the data structures definitions module 86 may be implemented in other devices of the network 18 and communicated to the applications software 30B. Before describing FIG. 5, which illustrates example building blocks for generating a predetermined quantity of statements for a notification system, an explanation of an example statement is first described below. As used herein, the term “statement” is defined as health-related information of an individual. In one or more embodiments, a statement may comprise one or more of the following: (a) a first comparison description of the one or more measurements between two objects in a temporal space; (b) a second comparison description of the one or more measurements between two objects in a user space; (c) an extrema description of the one or more measurements; (d) an interaction description of the one or more measurement; or (e) a trend description of the one or more measurements. The temporal space relates to a time period during which the measurements of parameters of the individuals are collected. The temporal space comprises one or more objects, each corresponding to a time interval, for example, “Monday” corresponding to a 24-hour long time interval, “morning” corresponding to a segment of a 24-hour long time interval, “workday” corresponding to a combination of the 24-hour long time intervals, “at work” corresponding to a combination of the segments of the 24-hour long time intervals, etc. The user space relates to one or more groups of individuals from which the measurements of parameters of the individuals are collected. The user space comprises one or more objects, each corresponding to a group of individuals, for example, “women in their 30′s,” “legal professionals,” “high school students,” etc. The extrema description describes one or more extreme observations across the temporal space and/or the user space based on the one or more measurements, for example, “a best running performance is achieved on the afternoon of Thursday.” The interaction description describes correlations between the one or more measurements across the temporal space and/or the user space, for example, “After workday, your active minutes are higher than your daily average.” The trend description describes the measurement trends observed across the temporal space and/or the user space, for example, “Today, your exercise duration is longer than any day in the last week.”

The template building component 92 (FIG. 4) is configured to build a set of templates that can be applied to generate the statements. The template building component 92 defines one or more building blocks and explores plural (e.g., all) possible combinations between the one or more building blocks. Referring now to FIG. 5, the one or more building blocks include a profile block 112 pertaining to long time intervals, a segment block 114 pertaining to short time intervals, a measurement block 116 pertaining to the measurement models, and a user block 118 pertaining to user groups. The profile block 112 defines one or more 24-hour time intervals such as, Monday, Tuesday, today, yesterday, a week ago, etc. In some embodiments, the profile block 112 defines one or more combinations of the 24-hour long time intervals based on information related to an individual. For example, the profile block 112 defines “work day” as any day between Monday through Friday, “slept-well day” as the days when the individual's sleep quality is above a threshold, etc. The segment block 114 defines one or more segments of 24-hour time intervals, for example, morning, before work, during commute to work. The measurement block 116 defines one or more measurement models pertaining to parameters. The parameters may indicate at least one of physiological or psychological signs of the user or physical activities of the user such as heart rate, respiration rate, running, cycling, etc. In some embodiments, for the parameter of heart rate, three measurement models are defined as average heart rate, resting heart rate, and maximum heart rate. In some embodiments, for the parameter of walking, four measurement models are defined as step count, average walking speed, walking duration, and walking distance. The user block 118 defines one or more user groups. In some embodiments, the user groups may be defined based on locale information, age information, professional information, social networking information, etc.

It should be appreciated that the examples of profile block 112, segment block 114, measurement block 116, and user block 118 as illustrated in FIG. 5 are for illustrative purpose only. The present disclosure is not intended to be limiting. As a running health program is intended to provide users with an accurate and comprehensive assessment of their health conditions, the template building component 92 (FIG. 4) may define any length of time intervals to be included in profile block 112 and segment block 114. The template building component 92 may further define more measurement models and refine user groups such that more detailed information can be conveyed to the users. It should also be appreciated that the template building component 92 may define additional building blocks to be applied to generate the statements. In some embodiments, the template building component 92 may define one or more additional profiles pertaining to an object, where the object may include a location, a particular type of device, a friend of the user, a community that the user belongs to, eating behavior, dressing behavior, etc. For example, a locale-based building block may be applied to provide users with performance assessments on different geographic locations (e.g., a user's marathon performance may vary in Washington D.C. vs. in Phoenix, Arizona). In yet another example, the one or more additional profiles may indicate that the user wears one particular brand-name or type of shoes longer than wearing another brand name shoe or type of shoe in a day. In yet another example, the one or more additional profiles may indicate that the user had a hamburger yesterday for lunch and had a cup of soup for lunch today. It should be appreciated that the above examples are for illustrative purpose only, and the present disclosure is not intended to be limiting.

Given the pre-defined building blocks, the template building component 92 (FIG. 4) explores plural (e.g., all) possible combinations between the pre-defined building blocks. The template building component 92 further refines all of the possible combinations based on certain criteria, for example, to exclude combinations that compare Mondays to Mondays, etc. In some embodiments, the template building component 92 may build a set of general templates that can be applied for all parameters. In some embodiments, the template building component 92 may build an individual set of templates to generate statements pertaining to a specific parameter.

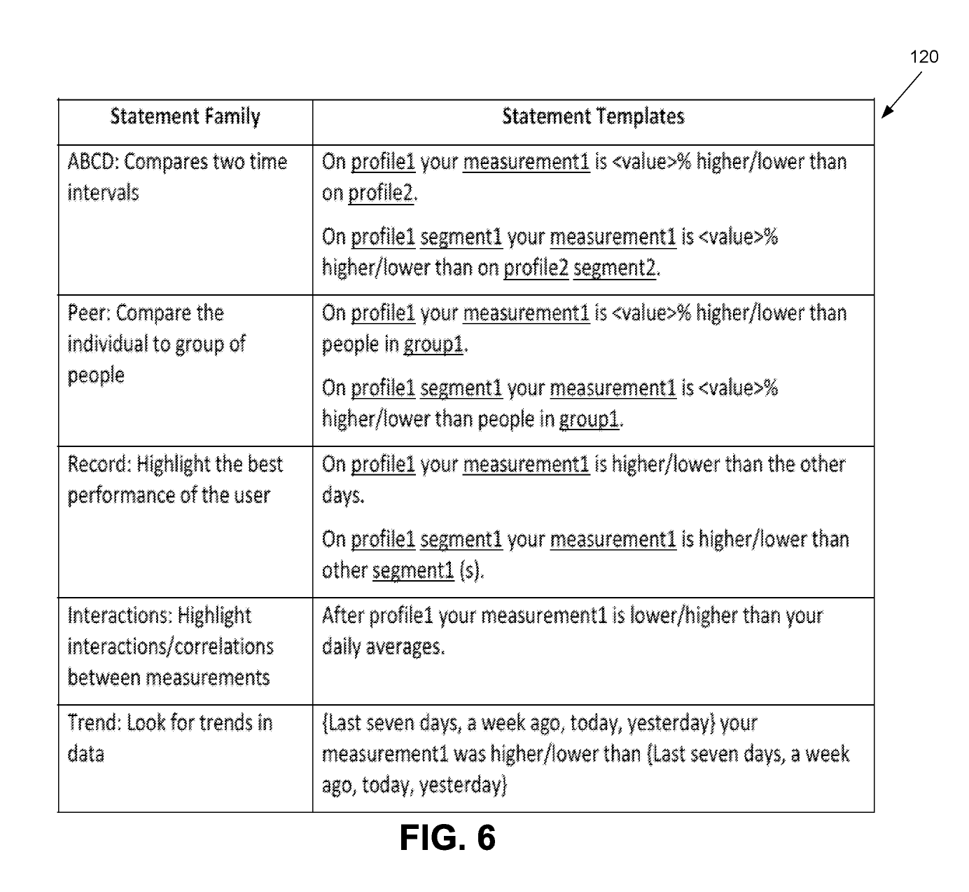

Referring to FIG. 6, which is a schematic diagram that illustrates an example statement family with one or more templates for a notification system, a plurality of templates 120 are illustrated in this example as built with respect to “measurement 1.” The plurality of templates 120 may be classified into plural (e.g., five) categories: (a) a category that compares one or more values related to “measurement 1” in two time intervals; (b) a category that compares an individual to a user group; (c) a category that highlights the best performance of the individual; (d) a category that highlights interactions and/or correlations between multiple values of “measurement 1;” and (e) a category that observes the trend in “measurement 1.” It should be appreciated that the templates and the categories of templates are for illustration purpose only. The present disclosure is not intended to be limiting. Other criteria such as locale information may be applied to classify the plurality of templates. In some embodiments, users of the program, including a coach, may also define the plurality of templates.

The data processing component 94 (FIG. 4) is configured to process the data received via the I/O interfaces 74 (FIG. 4) so that reliable measurements are used to generate the statements. Data received via the I/O interfaces 74 are information collected from one or more sensors implemented on the wearable device 12 (FIG. 2) and/or the electronics device 14 (FIG. 3). The data may also be sourced from elsewhere, such as from data structures storing user or population data and/or other devices. The data collected from the sensors may comprise all types of noise signals from the surrounding environment and/or from other sources that affect the accuracy of measurements. For example, a noise signal may magnify a measurement of heart rate to an unreasonable level and cause an erroneous measurement. In another example, noise signals may cause the loss of measurements that are continuously collected in real-time. The data processing component 94 may detect and correct the erroneous measurements, and recover the missing measurements based on one or more digital signal processing algorithms such that reliable measurements are provided to generate the statements.

In some embodiments, the data processing component 94 (FIG. 4) computes representations of daily exercise measurement data, for example, average values of the measurements in different daily segments. The data processing component 94 divides a day into semantically meaningful segments that can be referred to in the statements. Exemplary segments may comprise the time period during which the user is commuting to work, the time period during which the user is at work, or the time period during which the user is in a fitness club, etc. In some embodiments, the temporal segmentation is determined based on the location change of the user during the day. Location data may originate from a global positioning system (GPS), terrestrial radio frequency (RF) sources such as Wi-Fi, GSM, or near field communication (NFC), etc. Location data may comprise global coordinates of locations and/or names of the places. Location data may be collected via one or more applications implemented on the devices 12 and/or 14, for example, Moves app. Moves app produces two types of location data. The first type of location data contains a list of locations where the user has stopped for one minute or more. These places get a unique ID and additional attributes such as semantic information, address, and visit counts. The second type of location data contains data points collected over a movement trajectory during an activity. The activity may include cycling, walking trip, or transport, which typically starts from one place and ends in another (or the same) place. The second type of location data has no attributes, but the entire activity may have a classification based on transportation modality, step counts, and/or other measures. The notification system may classify the location data into four groups including home, work, other places, K-places (which denote intermediate places during commuting) based on one or more heuristic rules. For example, the heuristic rules may include (a) the place where the user spends the night is home; (b) the place where the user is in weekdays between 10 AM and 3 PM for more than 2 hours is work; (c) the places where the users stops between home and work is K-place. It should be appreciated that the examples described above are for illustrative purpose, and the present disclosure is not intended to be limiting. The temporal segmentation may be based on blind segmentation and the classification of locations may be based on the measurements and/or additional user metadata. In some embodiments, additional user metadata may be collected via interviewing, user input from a graphical user interface, answers on questionnaire, and/or other methods. In some embodiments, the classification of locations and the consequent segmentation of time periods may be trained using machine learning algorithm over a large population of data.

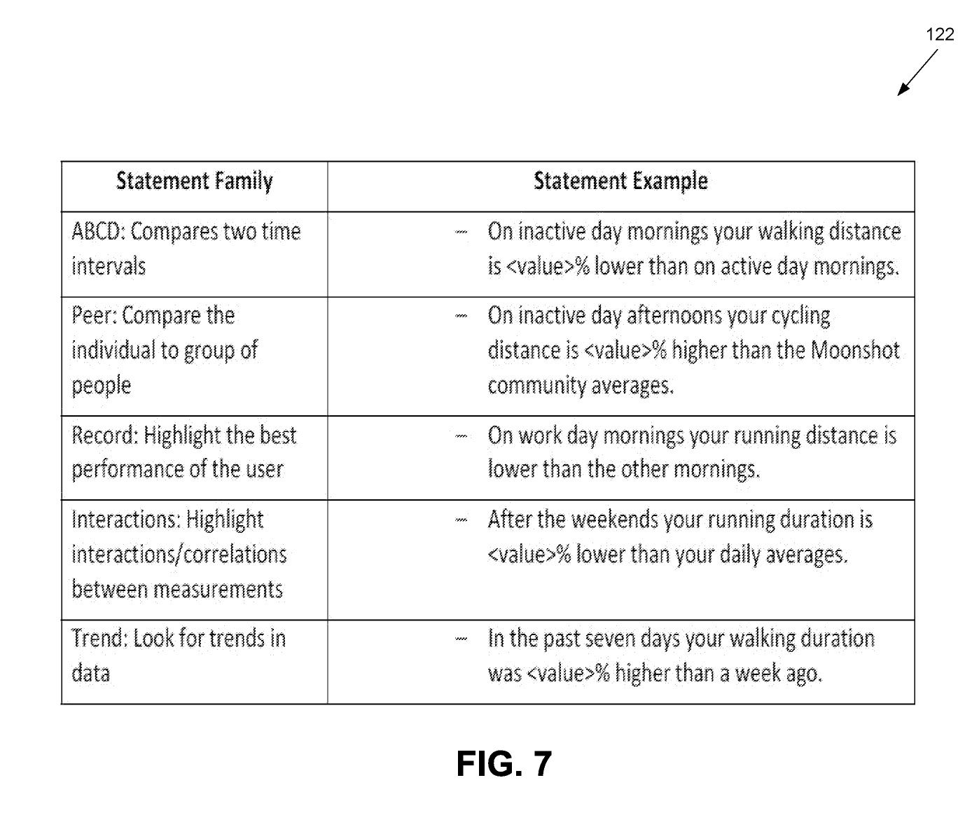

The statement generating component 96 (FIG. 4) is configured to generate one or more statements based on the measurements of parameters and the templates. In some embodiments, the measurements that are collected in real-time over a time period are further processed to generate augmented measurement sets. For example, measurements of the heart rate of a user over one month comprise a large amount of individual measurements. Augmented measurement sets may be generated to include an average heart rate over the one month, an average heart rate during sleep, a percentage of times when the heart rate exceeds 130, etc. Referring to FIG. 7, which illustrates an example of the generated statements based on the templates shown in FIG. 6, with respect to the parameter “walking,” two statements are generated as “On inactive day mornings, your walking distance is <value>% lower than on active day mornings;” and “In the past seven day, your walking duration was <value>% higher than a week ago.” Contrast to existing programs where a statement may simply summarize the walking distance in a daily and/or weekly basis, the illustrated statements comprise a comparison observation and a trend observation based on the large amount of measurements. The statements therefore, provide analytical assessments on the user's walking performance, and help the user to better capture the improvement by continue walking. It should be appreciated that the statements and the categories of statements in FIGS. 6-7 are for illustration purpose only. The present disclosure is not intended to be limiting. All combinations of the profile block 112, segment block 114, measurement block 116, and user block 118 shown in FIG. 5 can be applied to generate a statement.

Due to the large amount of available templates, the number of generated statements may be large. Even though an individual family may set a number of statements for presentation, the level of meaningfulness of the statements varies in accordance with the templates. For example, a statement of “In the past seven days, your walking duration was 20% higher than a week ago” is more meaningful than a statement of “On inactive day mornings, your walking distance is 30% lower than on active day mornings.” Presenting the number of statements based on the levels of meaningfulness helps the user to learn useful information more efficiently. The ranking component 98 (FIG. 4) is configured to compute a score via the truth engine 100 (FIG. 4) for each generated statement and rank all generated statements based on the scores. In some embodiments, the score of a statement indicates a level of truthfulness of the statement. The higher the score, the more accurate and/or insightful the information is conveyed via the statement. In some embodiments, the score of a statement indicates a level of interest or usefulness of the statement to the user. The higher the score, the more interesting or more helpful the statement that the user considers. In some embodiments, the score is computed using a same configuration of algorithms and/or parameters for all generated statements. In some embodiments, the score is computed differently for different families. In some embodiments, all statements generated by the data structures definitions module 86 (FIG. 4) are used by the chain building module 88 (FIG. 4). In some embodiments, only the highest ranked statements are used by the chain building module 88, or a subset of the list of statements that are ranked (e.g., top two). In some embodiments, additional information may be used for selection of the statements, including relevance to a particular health program (e.g., coaching program).

Many statements contain a number x which may represent an absolute measurement value, a difference between values, or a computed value using the truth engine 100 (FIG. 4). In some circumstances, the number x may appear incorrect in a statement. For example, the number x refers to tiny step counts or distances in a statement or the number x refers to a calorie burn 99% less than a typical user when doing a same exercise. Most of the incorrect measurements are due to the errors during sensing or missing information during transmission from one of the user devices (e.g., wearable device 12 or electronics device 14, FIG. 1) to the computing system 20 (FIG. 1). To eliminate the odd statements with erroneous measurements, the truth engine 100 defines a range [x_{bot,m}, x_{cell, m}] for the number x such that measurement value falls outside the range is filtered out for presentation.

In one embodiment, the score is computed based on statistical significance with four factors implemented therein. The four factors comprise: (1) Statistical significance of the difference based on the distributions and values D_{ab}; (2) Weight based on the number of occurrences of the referred context (e.g., element in profile block 112, segment block 114, measurement block 116, and user block 118 of FIG. 5) W; (3) Quality of data which contains the amount of missing data and measurement errors Q; (4) Custom weighting for each family U_{f}. To compute the statistical significance, a difference between two scalar measurement values xₐ and x_{b}, two probability density functions fₐ(x) and f_{b}(x), or the combinations thereof, may be implemented to represent a divergence value. In some embodiments, a Hellinger divergence measure is used to compare two probability density functions. The Hellinger divergence measures the squared difference between squared roots of the distributions as the divergence value:

$$\begin{matrix}{H}_{ab}^{2} = \frac{1}{2} \int {( \sqrt{{f}_{a} ( x )} - \sqrt{{f}_{b} ( x )} )}^{2} dx \\ = \frac{1}{2} \int {f}_{a} ( x ) + {f}_{b} ( x ) dx - \int \sqrt{{f}_{a} ( x ) {f}_{b} ( x )} dx \\ = 1 - \int \sqrt{{f}_{a} ( x ) {f}_{b} ( x )} dx\end{matrix}$$

In an embodiment where xₐ and x_{b} are discrete distributions, the divergence value H²_{ab} corresponds to the Euclidean distance between the two discrete distributions.

In another embodiment where xₐ and x_{b} are normal distributions N(μ, σ), the squared Hellinger divergence measure H²_{ab} is computed as:

$${P}_{ab} = \vert {H}_{ab}^{2} = 1 - \sqrt{\frac{2 {σ}_{a} {σ}_{b}}{{σ}_{a}^{2} + {σ}_{b}^{2}}} {e}^{- \frac{{( {μ}_{a} - {μ}_{b} )}^{⋀} 2}{4 ( {σ}_{a}^{2} + {σ}_{b}^{2} )}}$$

The divergence value falls in a range of [0, 1]. If two distributions are identical, the divergence value is 0 and if two distributions are non-overlapping, the divergence value is 1.

In some embodiments where a scalar measurement is compared to a distribution, the divergence value is obtained directly from the distribution function evaluated at the given measurement data point. If the distribution is a normal distribution N(μ, σ), the divergence value is computed as:

$${V}_{ab} = 1 - {e}^{- \frac{{( x - {μ}_{a} )}^{2}}{2 {σ}_{a}^{2}}}$$

In some embodiments where the comparison is performed between two scalar measurement values xₐ and x_{b}, the divergence value is computed as:

$${M}_{ab} = 1 - \frac{2}{1 + exp \frac{{( {x}_{a} - {x}_{b} )}^{2}}{{d}_{m}}}$$

where dₘ is determined based on the pre-defined range [x_{bot,m}, x_{cell,m}], e.g., dₘ=x_{cell,m}−x_{bot,m}.

The statistical significance Dab may be represented as:

$${D}_{ab} = \{ \begin{matrix}{P}_{ab} , & if both objects are distributions \\ {V}_{ab} , & if one object is a distribution \\ {M}_{ab} , & if both objects are scalars\end{matrix}$$

As the measurement distributions do not contain a number of occurrences of the object, an additional weighting may be applied. When the smallest count of the object occurrences in a given object pair is c, the weight term is computed as:

$${W}_{k} = 1 - {e}^{\frac{c}{α}} / β$$

where typical parameters are α=3, β=2.

The data quality Q is a scalar value in the range of [0, 1] indicative of the percentage of complete and correct measurements.

In some embodiments, each family may have a priori weight Uf applied to all the statements in the family. In some embodiments, each individual statement may have a specific priori weight.

The score is computed as a product of the individual four factors shown as:

Sk=D_{ab}WQU_{f}

Ranking component 98 (FIG. 4) is further configured to sort the statements in a family based on their computed scores in a descending order. It should be appreciated that the score computation described above is for illustrative purpose. Other factors may also be considered to compute the score of a statement. For example, the user's feedback on a specific type of statement may indicate the popularity of the specific type of statement, and thus, may influence the score of the statement. Other factors such as financial aspects may also affect the score of a statement. In some embodiments, one or more combinations of the factors may also be considered as a weighted factor for computing the score of a statement. The computation of statistical significance Dab set forth above is employed to those families where the statements highlight a difference in context such that a higher score is obtained if the measurements are different. In some other embodiments where the contexts are similar and a low score is obtained if the measurements are different, for example, “You are equally active on Mondays and Tuesdays,” the statistical significance D_{ab} is replaced by 1-D_{ab} for the ranking purpose. In some embodiments, alternative divergence measures may also be used to compare two probability density functions such as, Kolmogorov-Smirnov test, Kullback-Leibler measure, or the &-squared (e.g., Pearson) divergence measure. Therefore, the present disclosure is not intended to be limiting.

The ranked statements, or in some embodiments, unranked statements, are stored in the notifications data structures 84 and accessed (e.g., received by) the chain building module 88 (FIG. 4) for labeling (e.g., as starting statements, intermediate statement, or ending statements) and for establishment of predetermined possible paths by a content author, after which the execution component of the chain building module 88 commences.

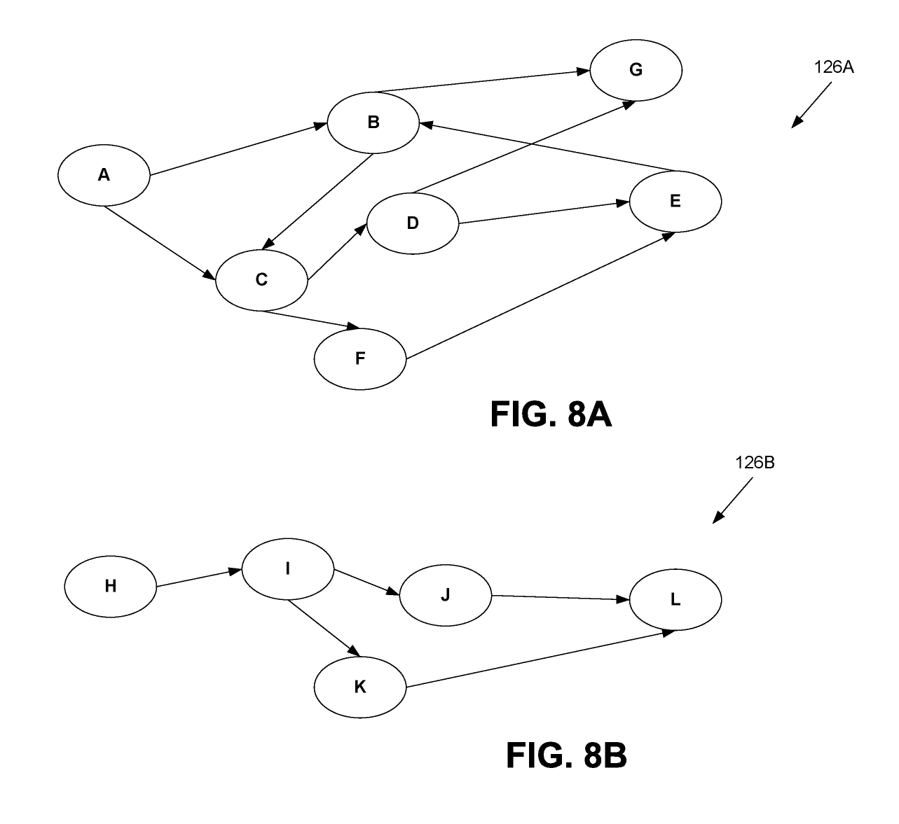

Having described certain functions of the data structures definition module 86 (FIG. 4), attention is now directed to functionality of the chain building module 88 (FIG. 4), which receives the data structures from the data structures definition module 86, associates the statements to nodes comprising starting, intermediate, and ending statements, establishes predetermined pathways among the nodes for publishing the statements, and builds a chain of statements, each statement presented in non-overlapping intervals to the user to collectively form a narrative. FIGS. 8A and 8B are schematic diagrams of example possible paths or pathways 126A and 126B, respectively, of starting, intermediate, and target nodes with statements that are established by a notification system. In one embodiment, the possible paths are established by a content author (e.g., based on his or her observation or intuition) in conjunction with the pathway establishment component 104 (FIG. 4) based on the associations (labeling based on his or her observation or intuition) performed by the content author in association with the node association component 102 (FIG. 4). Referring to FIG. 8A, the starting node comprises notification or statement “A”, and the two target nodes comprises notifications or statements G and E (the letters representing statements). Hereinafter, reference is made to statement, with the understanding that the terms statement and notification are used interchangeably. Generally, the intermediate nodes comprise short-term trends related to the same context and measures as in a previous node. The target nodes are positive opposites of the starting nodes, generally providing positive end points from intermediate nodes (though in some circumstances, may reveal where the user is coming up short). For purposes of illustration, the statement “G” represents a scenario where the user has met his or her goal (e.g., health goal), and “E” represents a scenario where the user is behind his or her goal. The network of intermediate nodes comprise the statements B, C, D, and F. As described earlier, all of the possible statements are predetermined in number, labeled, and then plural (e.g., all) possible paths from the starting node to the target nodes are established. From the depicted example, after the starting node A, possible paths to the next node include intermediate nodes B and C as the next nodes. As explained above, these possible paths are indicated by the statement table of the node from which the next node transition occurs. From node B, the next node may be the target node G or the intermediate node C. From node C, possible next nodes that define points in the path may be nodes D or B. Possibilities from the other nodes are also illustrated. As described above, which of the next nodes to select is determined based on the updated measures of the respective statement table computed by the measure compute component 106 as or based on input data received. The starting node A is also referred to as an opportunity. The starting node A also comprises an entry point for a chain, the entry point determined in one embodiment based on measures or scores computed by the measures compute component 106 for all labeled starting nodes. For example, the start of a chain is triggered if a confidence score is, for example, higher than a predetermined threshold (e.g., 80%) and comprises the highest score among the possible starting nodes.

In some embodiments, the starting node may be determined based on user response to a prompt delivered by the notification system. For instance, the user may be informed about the detection of an opportunity in a dashboard and asked if he or she wants to start a personal program module that helps to improve the lifestyle in the referred context.

Similarly, in FIG. 8B, the possible paths 126B established by the content author in conjunction with the pathway establishment component 104 (FIG. 4) are illustrated. The starting node comprises a statement H, followed by intermediate node with statement I. From the intermediate node comprising statement I, the paths continue to the nodes with statements J and K, followed respectively by a target node comprising a statement L indicating that the user has achieved his or her goal. Processing as described in association with the path 126A of FIG. 8A is similarly applied to the path 126B, and hence omitted here for brevity.

FIG. 9 is a schematic diagram that illustrates an example of candidate (possible) chains or pathways of notifications 126C used by an embodiment of a notification system, further illustrating the types of content for the paths generated by the notification system (e.g., similar to the paths 126A, 126B). The left-most node is a starting node, and comprises the statement, “On weekends you are less active than weekdays. Can you change that?”. The nodes on the right comprise possible target nodes, and from top target node to bottom target node include the statements, “On weekends you walk N minutes more than on weekdays. Nice!”; “On Saturdays you have more active minutes than any other day. Well done!”; “In this month your cycling distance on weekends has increased 90%. Wow!”. The intermediate nodes (shown in the middle “column” of FIG. 9) represent intermediate short-term steps between the starting node and the target nodes. The intermediate nodes, shown coupled to the starting node and target nodes, reveal a multitude of the possible or candidate paths. Statements from the top intermediate node to the bottom intermediate node include, “Last weekend you walked N % more than usual”; “This Saturday you had N active minutes more than last week.”; “This weekend your cycling distance was N % more than in a typical weekend.” The plural possible chains or paths (e.g., as connected according to the directed arrows from the start, intermediate and end nodes) are represented by lists of statement IDs in statement tables associated with each of the start and intermediate nodes.

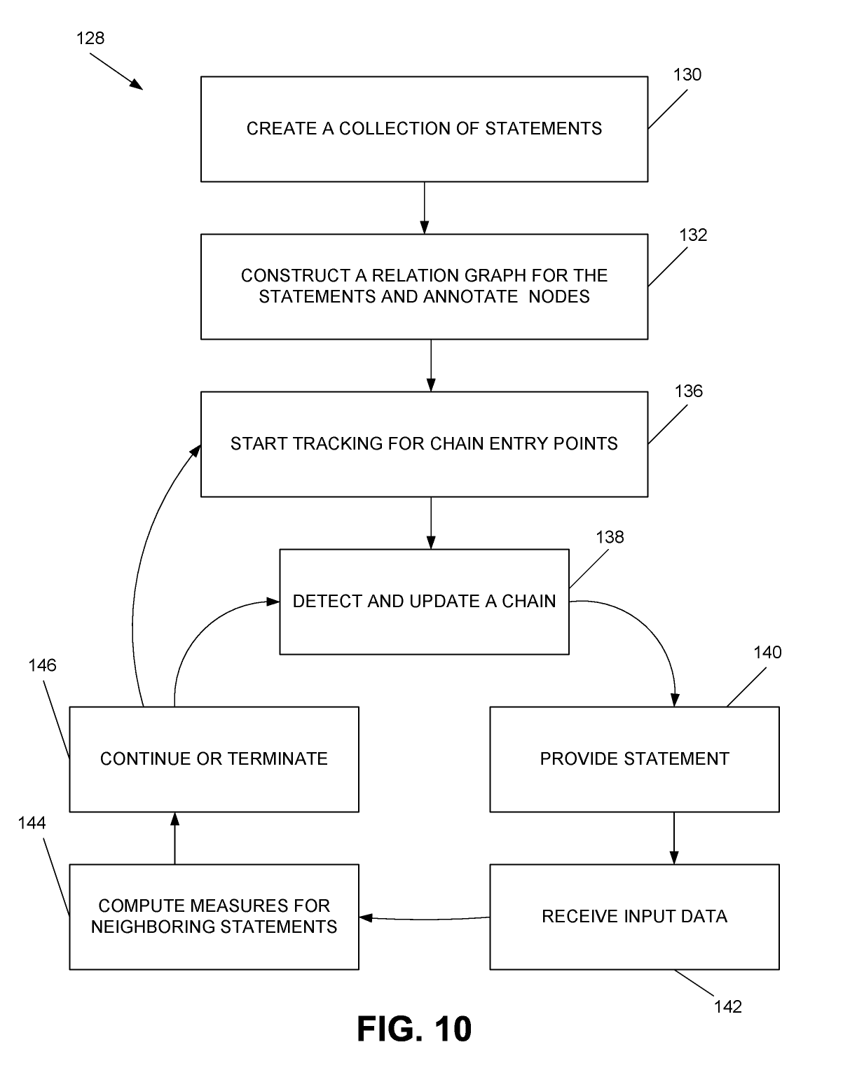

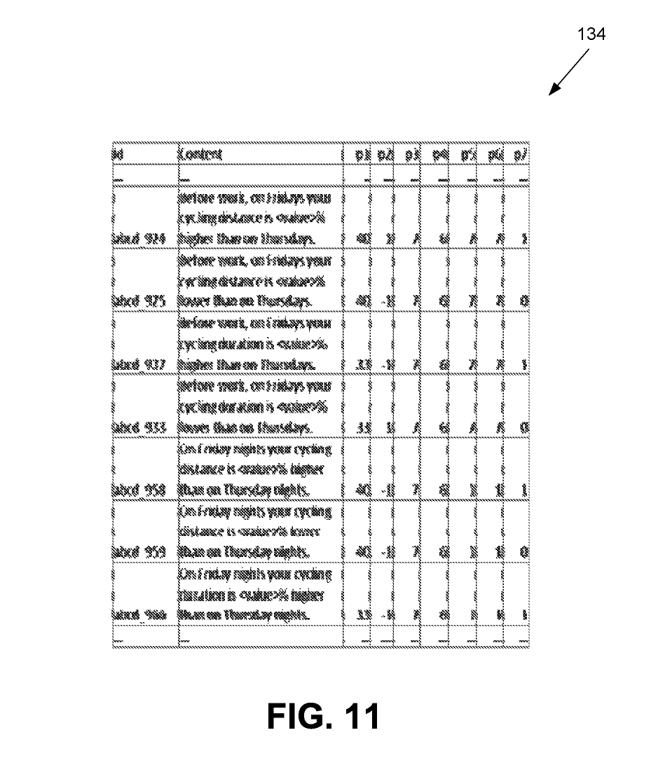

FIG. 10 is a schematic diagram that illustrates an example processing model 128 for path tracking for an embodiment of a notification system. In one embodiment, the model 128 (or equivalently, algorithm) is implemented by the application software 30B (FIG. 4), and hence reference hereinafter to the model 128 performing steps in a process are to be understood as being executed by the processing circuit 70 and software code of the application software 30B. The model 128 includes creating a collection of statements (130). As noted above, the data structures definition module 86 (FIG. 4) generates the statements based on a coding of the statements. For instance, a large collection of statement candidates are generated which address all detectable contexts, measurements and their statistical relations. The processing is based on data from wearable sensors, applications running in a device (e.g., a smartphone), separate measurement devices such as weighting scales or air quality sensors, usage of connected services such as social media, data from questionnaires, and other interaction with the service, and demographic information about the user. The model 128 then constructs a relation graph or candidate chain for the statements and annotates the nodes (132). In one embodiment, a content author in association with the node association component 102 associates the nodes with starting, intermediate, and ending statements, and the content author in association with the pathway establishment component 104 establishes all possible pathways among the nodes and also creates statement tables (or lists) for the starting and ending nodes. The statement table comprises all possible nodes (statements) that can possibly follow the node that contains the statement table. Each statement is associated with a unique ID and parameters that describe the context, measurement, and direction (e.g., a categorical variable with a predefined meaning (e.g., lower=1) and that gives the verse of comparison between measurements) of the statement. An example list of the statements is shown in FIG. 11 as a statement table 134. As illustrated in the statement table 134, the scoring of each statement is based on the parameters. A statement scoring algorithm collects the statistics of the measurements represented by the parameters and computes a unique score for each statement. In one embodiment, the value of the score is between 0 and 100. The model 128 starts tracking for chain (chain) entry points (136). As noted above, the entry point may be based on the measures (e.g., scoring), and in some embodiments, the entry point may be based on complete chains and/or based on experiences of others. The model 128 detects and updates a chain (138) and provides a statement corresponding to the node that is selected as part of the chain (140). For instance, if the detection is of a starting node (e.g., based on the highest score among all of the starting statements), the model 128 establishes the start of a possible pathway or chain. The model 128 then provides the statement for publication (140). The model 128 then receives input data (142) and computes measures for neighboring statements (next statements) of a statement table of the node for the published statement (144). For instance, the statements (neighbor statements) are in the statement table and identified by an ID and parameters that are scored. The model 128 continues or terminates (146). For instance, the scores may not meet a predetermined threshold after a predetermined time has elapsed, causing a termination event and processing to commence at (136) for a new chain start. Alternatively, the scores may reach a predetermined threshold, and the highest score of a statement from the published statement table is used as a basis for selecting the next node (statement) (138), after which the statement is published (142) and so on.

In one embodiment, the progress pace of the user towards the target node (e.g., goal) is leveraged to decide whether to stop the chain or speed the node progression (e.g., the progress evaluated by the measurements and the time span with respect to a previous statement). In particular, if the progress from one node to the next generated one is too little and too slow, the chain may be stopped, especially if other similar opportunities are triggered. Conversely, if the user quickly follows the triggered path and more statements are generated with substantial progress, the chain could be given more importance by, for example, focusing on that one and removing any similar chain in embodiments where chains are generated concurrently.

In some embodiments, the user's progress and motivation can be detected from the increase of candidate nodes showing that the user is following that path since his or her behavior is creating new observations within that path. Such knowledge may be used to detect preferences of a user for future chain and node selection and/or to trigger or suggest a program (e.g., coaching program) to influence behavior change.

In some embodiments, the chain construction (e.g., at 132 in FIG. 11) may be based on evaluating aggregated measurement data during a predefined baseline period. The measurement data is processed and opportunities are identified to improve certain aspects in the lifestyle of the end-user. Inferred opportunities may refer to certain (recurring) lifestyle behaviors which may cause health issues in the long term or identified immediate, short term or long term health risks. Health issues may relate to, for instance, achieved physical activity levels, current physical fitness, blood pressure, air quality, and the like. From the inferred opportunities, specific and concrete starting nodes are created, as well as intermediate trend and context related nodes, and target nodes that reflect certain successes/achievements. The created chain is basically constructed from a subset of all possible start, intermediate and target-nodes. In some embodiments, a chain is constructed on the basis of a certain end-user profile that is classified/inferred from the measurement data. Each profile category has its own predefined (template) chain comprising starting, intermediate and target nodes. Selection of a starting node may be based on a scoring mechanism.

In some embodiments, to maximize the success of reaching a target node (or optimize the path from the starting node to the target node), the model 128 may assign weights to certain node transitions (especially in cases where multiple motivation/feedback/advice statements can be provided to the user). The weights are learned through experiences from previous users where similar (sub)chains were applied. More concretely, to assess the effect/rate of success of certain path transitions, the model 128 may request feedback from the user to score the usefulness of previous statements that were shown a consequence of reaching certain nodes in the chain (e.g., somewhat similar to a social media “like” button). Node transition weights may be derived from the scores provided by the user. Moreover, the scoring may be used to develop new/updated current chains that target specific goals.

Reference is now made to FIGS. 12A-12C, which illustrates an example embodiment of a method for chaining statements as implemented by the chain building module 88. FIG. 13A shows schematically the nodes with the corresponding statement to be published and statement table. FIG. 12B provides an illustration of an example timeline for publishing the statements. FIG. 12C illustrates the possible paths from each node as indicated by the statement tables. Referring to FIGS. 12A and 12C, a first node (start node (0)) comprising statement 0 is selected based on having a highest score among a plurality of starting statements that meet or exceed a predetermined threshold. Through selection of the start node (0), the statement 0 is published at, for instance, Day 1 as illustrated in FIG. 12B. The start node (0) comprises a statement table shown in FIG. 12A that lists potential nodes from the start node to a next node, and in that sense, potential paths to follow from the start node (0). That is, the statement table comprises as possible next nodes, node 1 (statement 1), node 2 (statement 2), node 3 (statement 3), among others. These potential nodes reveal paths from the start node (0), which is similarly illustrated in FIG. 12C (e.g., the potential nodes from start node (0) are nodes 1, 2, or 3. Between the publishing of statements from nodes, input data is received. The data may be received continuously, intermittently, or as scheduled (e.g., during predetermined uploads from a user device). The statements from the statement table of the start node (0) are scored, with the highest score that meets or exceeds a predetermined level or value being the statement and corresponding node that is selected. In this example, as illustrated in FIG. 12A, the node (2) from the statement table of the prior start node (0) is selected, and published after a given interval of time from the last statement publication (e.g., a week from Day 1) as illustrated in FIG. 12B. Node (3) also comprises a statement table that includes nodes 11-13 (among others) as possible next nodes (subsequent to node (2)), as reflected by the node arrangement in FIG. 12C (e.g., node (2) is linked to nodes 11, 12, and 13). After another week of data input, a next node is selected based on scoring of the statements of the statement table for node (2), the scoring based on the input data received between week 1 and week 2. In this example, statement 13 publishes (as illustrated in FIGS. 12B and 12C), and the process continues with receiving input data during the next interval (e.g., next week) and computing scores for the statements of the statement table to determine the highest score for publication. The process continues until the publication of a statement for an ending or target node or due to an occurrence of a termination event.

FIGS. 13A-13E are screen diagrams 148 (e.g., 148A-148E) that illustrate example notifications or statements presented to a user by a notification system in accordance with an embodiment of the invention. Each screen diagram 148 may be a user interface associated with the electronics device 14 or the wearable device 12. In general, the screen diagrams 148 present notifications or statements that, depending on their position in a chain, may be perceived as advice, motivation, or feedback. Note that each screen diagram 148 is shown with an ellipse symbol surrounding a letter beneath the statement, the ellipse not actually presented to the user but rather, provides a representation as to which node from FIG. 8A is being presented, thus facilitating an understanding of which nodes from the plural possible paths from FIG. 8A are being used to provide the narrative and in what order of presentation. However, in some embodiments, the text may be accompanied by graphical symbols. Referring to FIG. 13A, based on the input data received at the computing system 20 (FIG. 4) and the computed measures, the notification system causes the presentation in the screen diagram 148A the following statement: We noticed that you sometimes take a walk in the park close to your home. This kind of activity helps your heart if you perform it often.” At a non-overlapping time (again after data input and updated measures), the screen diagram 148B (FIG. 13B) presents the following: “You have taken the walk in the park this week more often than in the previous month. Can you stretch it 15 minutes longer?” Similarly, in another overlapping time interval, the screen diagram 148C of FIG. 13C presents the following statement: “The walk in the park is already the longest active part of your weekly routine. It helps you to meet your target weight two weeks early.” The screen diagram 148D of FIG. 13D presents later, “On days when you take the walk late in the evening, you fall asleep later. Don't start the walk after 19:00 and you'll be better prepared for sleep. Finally, the screen diagram 148E of FIG. 13E presents the following statement: “Your fitness, based on your heart rate information during the park walk, has improved in the last month. Well done! This is largely due to the walks.” When comparing to the possible paths 126A illustrated in FIG. 8A, the notification system has selected, based on the greater of measures taken at each node transition that meet or exceed a minimum threshold and based on the received user data over time, nodes in the following order: A, B, C, F, and G. The statements help to guide and motivate the user towards a heart-healthy goal in a logical, narrative fashion. Note that the example illustrated in FIGS. 13A-13E are merely illustrative, and that additional or fewer statements may be used in some circumstances of the same or different format. For instance, the notification system may interject additional comments and/or reminders to the user in some embodiments. In some embodiments, the presentation format may be more in the form of a dialogue, such as enabling a dialogue between the user and a virtual coach.

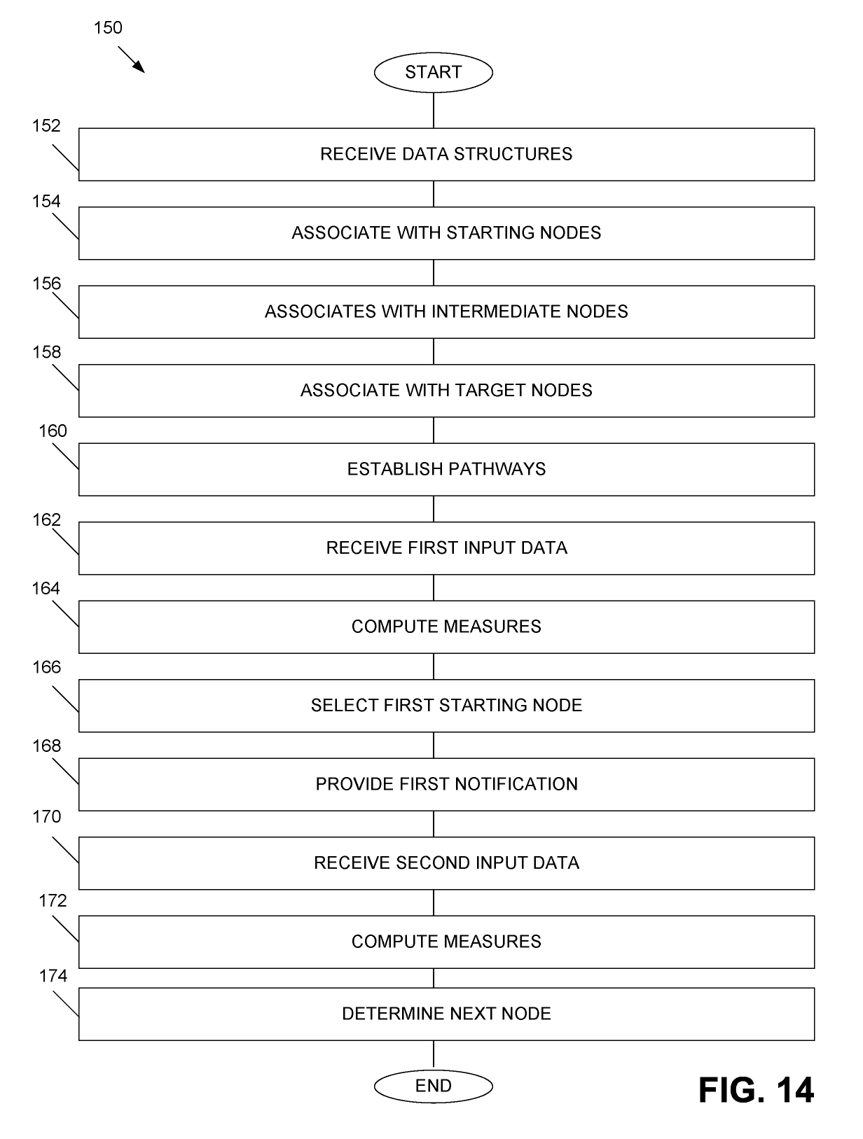

In view of the description above, it should be appreciated that one embodiment of a notification method (e.g., implemented by the notification system), depicted in FIG. 14 and referred to as a method 150 and encompassed between start and end designations, comprises receiving a predetermined quantity of data structures that are contextually related, the data structures each comprising a notification (152); associating a first plurality of the predetermined quantity of data structures with respective starting nodes (154); associating a second plurality of the predetermined quantity of data structures with respective target nodes (156); associating the starting nodes and the ending nodes with a network of intermediate nodes, the network comprising a third plurality of the predetermined quantity of data structures (158); establishing plural possible pathways from each of the starting and intermediate nodes, each of the starting and intermediate nodes further comprising a statement table, the possible pathways from each of the starting and intermediate nodes indicated in the respective statement tables (160); receiving first input data (162); computing respective measures for the first plurality of predetermined quantity of data structures based on the input data (164); selecting a first starting node among the starting nodes based on the computed measures (166); providing a first notification of the first starting node (168); receiving second input data (170); computing measures for the statement table of the first starting node based on the second input data (172); and determining a next node, from the statement table of the first starting node, to follow and link to the first starting node based on the computed measures for the statement table of the first starting node to provide a chain of notifications providing an indication of progress in advancing from the first starting node to one of the target nodes (174).

Any process descriptions or blocks in the flow diagram described above should be understood as representing modules, segments, or portions of code which include one or more executable instructions for implementing specific logical functions or steps in the process, and alternate implementations are included within the scope of an embodiment of the present invention in which functions may be executed substantially concurrently, and/or additional logical functions or steps may be added, depending on the functionality involved, as would be understood by those reasonably skilled in the art of the present invention.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For instance, in some embodiments, content selection (e.g., of the statement narrative) may be based on complete chains. For instance, referring to FIG. 8A, one chain may include nodes A to B to G, whereas another chain may be A to B to C to D to G, and so on as shown by the various directed arrows in FIG. 8A. In one embodiment, the chain determining component 108 (FIG. 4) may, from a given starting point, check if there is any incoherency between alternative chains. Two incoherent chains may be, for example, about increasing the cycling time and the walking time during commuting whereas the user disposes of a limited commuting time. If the chain determining component 108 determines there is an incoherency, the chain determining component 108 selects the chain involving that activity on which the user shows more flexibility in his or her lifestyle patterns. In one embodiment, flexibility may be measured as suggested by personal cost units (PCUs). For instance, if the PCUs of cycling during weekends (e.g., an activity associated with one of the statements in one of the chains) is lower than the PCUs on walking (e.g., an activity associated with one of the statements in the other of the chains), then the chain determining component 108 selects the intermediate node involving cycling. This algorithm facilitates selection of a chain involving a path that has more probability to reach a target node, given that it takes into consideration the preferences of the user. In some embodiments, such a probability may be explicitly obtained through learning from other users with similar lifestyles. For instance, the application software 30B (e.g., the chain determining component 108) of FIG. 4 may compute transition probabilities from one type of statement to another type of statement based on observed chains created by or formed for similar users. Based on the other user chains, the chain determining component 108 can easily select the path that maximizes the probability of reaching a meaningful target.

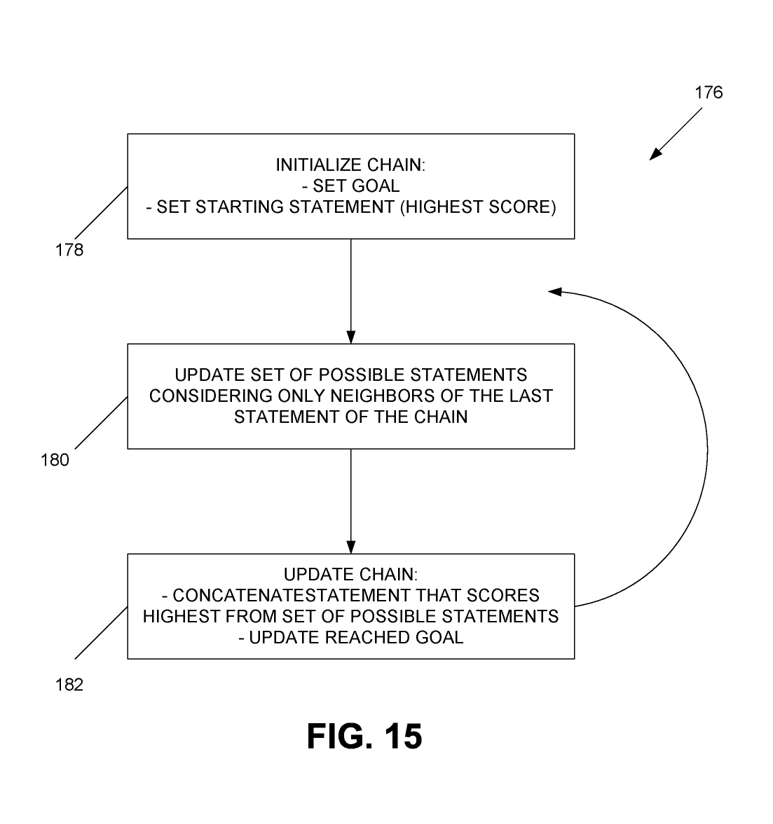

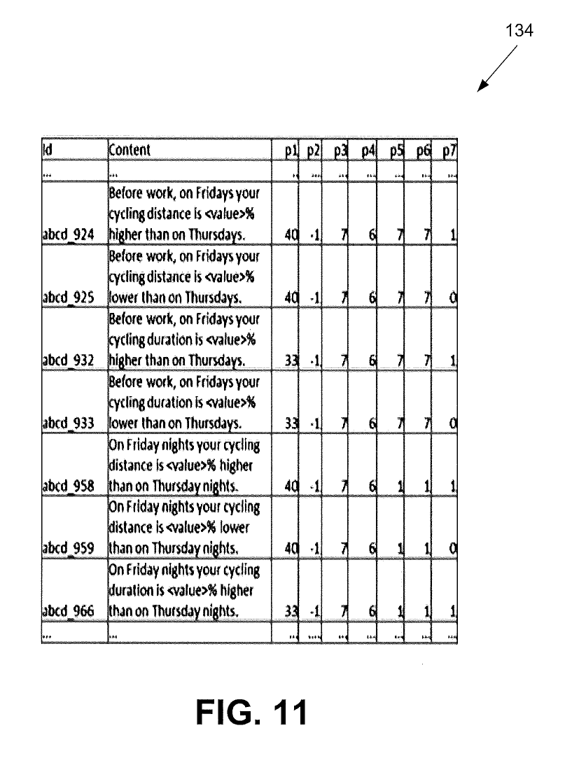

Note that in some embodiments, similar selection principles and/or methods may be used to limit the number of concurrent chains being triggered, even if those chains are coherent. One benefit to such an approach is it keeps a reasonable number of statements in the user's feeds. In some embodiments, computation of the scores of the chains may be achieved in one of a variety of ways. Referring to FIG. 15, shown is an example scoring method 176 that may be used by the application software 30B (FIG. 4) to compute the score of each chain. In general, the statement that scores highest at each step is the statement that will contribute to the score (achieved goal) of the chain. The method 176 selects a goal and the statement with the highest score between neighboring candidates (178). The Goalk is the total achieved goal from 0 (start of the chain) until step k. At step k, the system considers only the statements that are neighbors of the statements that scored highest the previous step (e.g., at k-1), and the statement that scores highest is the one that is selected by the method 148. At each step, the total score of the chain is updated (180). In one embodiment, a statement table (e.g., similar to statement table 134 of FIG. 11) may be used to derive the updated scores. The chain is updated, and statements that score highest from a set of all possible statements are concatenated, and the reached goal is updated (182). Consider the equation, Goalₖ=Σₜ₌₀^{k-1}Goalₜ where Goalₜ represents the goal achieved through the statement that scores highest at step t. This modality allows the method 176 to keep track of the progress due to the achievements that have highest confidence. In some embodiments, the Goal ₖ is computed from all the available measurements of each step of the chain from 0 to k as total progress. This modality considers all the progress of the user. In some embodiments, the notification system sets the goal that needs to be reached. The goal is linked to the detected chain (e.g., the opportunity to increase activity). When the end goal is reached, a statement may be presented to the user.

In some embodiments, the notification system computes the time and achievable goal of the chain. The notification system acts to help the user to achieve the settled goal by advising the user (e.g., increase activity on Sunday morning). The notification system can provide the user insights on how the user is progressing (e.g., “you are on track to achieve your goal next week!”). Input of the user may be kept into account during the process of settling the goal (and time) of the chain. A user can always define when to stop or to change the objective of his or her chain.

As another example of alternative embodiments, measures or scores may be also used to select nodes of the paths and to carve out from those paths a chain that ultimately is used as the narrative to the user. In another example embodiment, generation of statements may be prompted by a user request. For instance, if the input data comprises a request from the user to receive a report of the past week sleep quality, the processor 72 (FIG. 4) instructs the data processing component 94 (FIG. 4) to process the request from the user and provide one or more statements pertaining the sleep quality of the user in the past week.

Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

At least the following is claimed:

1. A system, comprising:
a first device, comprising:
a memory comprising instructions; and
a processing circuit configured to execute the instructions to:
receive a predetermined quantity of data structures that are contextually related, the data structures each comprising a notification;
associate a first plurality of the predetermined quantity of data structures with respective starting nodes;
associate a second plurality of the predetermined quantity of data structures with respective target nodes;
associate the starting nodes and the ending nodes with a network of intermediate nodes, the network comprising a third plurality of the predetermined quantity of data structures;
establish plural possible pathways from each of the starting and intermediate nodes, each of the starting and intermediate nodes further comprising a statement table, the possible pathways from each of the starting and intermediate nodes indicated in the respective statement tables;
receive first input data;
compute respective measures for the first plurality of predetermined quantity of data structures based on the input data;
select a first starting node among the starting nodes based on the computed measures;
provide a first notification of the first starting node;
receive second input data;
compute measures for the statement table of the first starting node based on the second input data; and
determine a next node, from the statement table of the first starting node, to follow and link to the first starting node based on the computed measures for the statement table of the first starting node to provide a chain of the notifications in narrative form, the chain of notifications providing an indication of progress in advancing from the first starting node to one of the target nodes.

2. The system of claim 1, wherein the processing circuit is further configured to execute the instructions to determine that the next node comprises one of the target nodes.

3. The system of claim 1, wherein the processing circuit is further configured to execute the instructions to determine that the next node comprises one of the intermediate nodes.

4. The system of claim 3, wherein each of the notifications comprise a statement, wherein the statement comprises a reference to user data and a behavioral goal of the user and optionally a user preference.

5. The system of claim 4, wherein the processing circuit is further configured to execute the instructions to:
determine the next node by selecting from the statement table of the first starting node a statement among a plurality of statements of the statement table that comprises one or any combination of a measure that meets or exceeds a threshold score and is a highest score among the plurality of statements, features that meet personalized criteria, features that meet historical criteria, or based on context aware features; and
provide the statement as a second notification, the statement of a second node, the first and second notifications collectively in narrative form and providing an indication of progress in advancing from the first starting node to the second node and ultimately to one of the target nodes.

6. The system of claim 5, wherein the processing circuit is further configured to execute the instructions to:
receive third input data;
compute measures for the statement table of the second node based on the third input data;
determine a next node, from the statement table of the second node to follow and link to the second node based on the computed measures for the statement table of the second node.

7. The system of claim 5, further comprising a second device communicatively coupled to the first device, the second device configured to receive the first notification at a first instance in time and the second notification at a second instance of time and present the first and second notifications in non-overlapping temporal intervals, wherein the second device is configured to present the first and second notifications in human-perceivable format.

8. The system of claim 1, wherein the first data and the second input data comprise user data and an associated context.

9. The system of claim 8, wherein the user data comprises data measuring at least one physiological parameter, movement parameter, or a combination of the physiological parameter and the movement parameter.

10. The system of claim 1, wherein the processing circuit is further configured to execute the instructions to select the first starting node based on the computed measure of the first starting node meeting or exceeding a threshold score and having a highest score compared to the other predetermined quantity of data structures.

11. The system of claim 1, wherein the processing circuit is further configured to execute the instructions to determine one or more next nodes based on tracking a parameter of the user data and continually computing the measures of the transitioned from node until either one of the target nodes is reached or a termination event is reached, wherein the termination event occurs when a transition from the first starting node or from an intermediate starting node has not occurred within a predefined period of time.

12. The system of claim 1, wherein the processing circuit is further configured to execute the instructions to adjust a rate of determining the next node based on a received indication of progress a user makes in achieving a goal.

13. The system of claim 1, wherein the processing circuit is further configured to execute the instructions to receive an indication of progress a user makes in achieving a goal based on the receipt of the additional user data, wherein the processing circuit is further configured to execute the instructions to determine a preference of the user for one or more of the notifications or suggest a program for the user based on the receipt of the indication of progress.

14. The system of claim 1, wherein the processing circuit is further configured to execute the instructions to determine the measures based on a similarity of stored notifications with a higher probability in enabling a user to reach a goal compared to other stored notifications or based on user feedback of a desirability for one or more of the stored notifications in reaching the goal.

15. The system of claim 1, wherein the processing circuit is further configured to execute the instructions to select the first starting node based on:
detecting an opportunity for behavioral change;
prompting a user to accept the opportunity; and
receive an indication that the user has accepted the opportunity or select based on user context.

16. The system of claim 1, wherein the processing circuit is further configured to execute the instructions to associate and establish based on content author input.

17. A method implemented by one or more processing circuits, the method comprising:
receiving a predetermined quantity of data structures that are contextually related, the data structures each comprising a notification;
associating a first plurality of the predetermined quantity of data structures with respective starting nodes;
associating a second plurality of the predetermined quantity of data structures with respective target nodes;
associating the starting nodes and the ending nodes with a network of intermediate nodes, the network comprising a third plurality of the predetermined quantity of data structures;
establishing plural possible pathways from each of the starting and intermediate nodes, each of the starting and intermediate nodes further comprising a statement table, the possible pathways from each of the starting and intermediate nodes indicated in the respective statement tables;
receiving first input data;
computing respective measures for the first plurality of predetermined quantity of data structures based on the input data;
selecting a first starting node among the starting nodes based on the computed measures;
providing a first notification of the first starting node;
receiving second input data;
computing measures for the statement table of the first starting node based on the second input data; and
determining a next node, from the statement table of the first starting node, to follow and link to the first starting node based on the computed measures for the statement table of the first starting node to provide a chain of notifications providing an indication of progress in advancing from the first starting node to one of the target nodes.

18. The method of claim 17, further comprising providing a second notification from the statement table of the determined next node, the first and second notifications configured for presentation in non-overlapping time intervals and comprising at least a portion of the chain.

19. A non-transitory computer readable storage medium comprising instructions that, when executed by one or more processing circuits, causes the one or more processing circuits to:
receive a predetermined quantity of data structures that are contextually related, the data structures each comprising a notification;
associate a first plurality of the predetermined quantity of data structures with respective starting nodes;
associate a second plurality of the predetermined quantity of data structures with respective target nodes;
associate the starting nodes and the ending nodes with a network of intermediate nodes, the network comprising a third plurality of the predetermined quantity of data structures;
establish plural possible pathways from each of the starting and intermediate nodes, each of the starting and intermediate nodes further comprising a statement table, the possible pathways from each of the starting and intermediate nodes indicated in the respective statement tables;
receive first input data;
compute respective measures for the first plurality of predetermined quantity of data structures based on the input data;
select a first starting node among the starting nodes based on the computed measures;
provide a first notification of the first starting node;
receive second input data;
compute measures for the statement table of the first starting node based on the second input data; and
determine a next node, from the statement table of the first starting node, to follow and link to the first starting node based on the computed measures for the statement table of the first starting node to provide a chain of notifications providing an indication of progress in advancing from the first starting node to one of the target nodes.

20. The non-transitory computer readable storage medium of claim 19, wherein the instructions, when executed by the one or more processing circuits, further causes the one or more processing circuits to provide a second notification from the statement table of the determined next node, the first and second notifications configured for presentation in non-overlapping time intervals and comprising at least a portion of the chain.
